(19) United States
(12) Patent Application Publication
(10) Pub. No.: US 2006/0269520 A1
(43) Pub. Date: Nov. 30, 2006

(54) XIAP therapy

(75) Inventors: Robert G. Korneluk, Ottawa (CA); Catherine Tsilfidis, Gatineau (CA); Peter Liston, Ottawa (CA); Alex E. MacKenzie, Ottawa (CA); George Robertson, Halifax (CA); Nathalie Gendron, Hull (CA); James Waring, Ottawa (CA); Doug Mahoney, Ottawa (CA)

Correspondence Address: CLARK & ELBING LLP, 101 FEDERAL STREET, BOSTON, MA, 02110, US

(73) Assignee: Children's Hospital of Eastern Ontario, Ottawa (CA)

(21) Appl. No.: 11/342,718

(22) Filed: Jan. 30, 2006

Related U.S. Application Data
(60) Provisional application No. 60/648,304, filed on Jan. 28, 2005.

(51) Int. Cl. A61K 48/00

(52) U.S. Cl. 424/93.2

(57) ABSTRACT

The invention features methods, compositions and kits for the treatment of degenerative disorders.

### CROSS-REFERENCE TO RELATED APPLICATIONS

This application claims benefit from U.S. Provisional Application No. 60/648,304, filed Jan. 28, 2005, hereby incorporated by reference.

### BACKGROUND OF THE INVENTION

Retinal degenerations are a major cause of irreversible blindness worldwide. Diseases affecting the retina such as retinitis pigmentosa (RP), glaucoma, retinal ischemia and age-related macular degeneration cause visual loss in millions of people in North America alone, and yet little is known about the mechanisms or causes of many of these diseases.

RP is a genetically heterogeneous group of retinal degenerations characterized by progressive night blindness, reduction or loss of visual acuity and constriction and gradual loss of the visual field. A decline in the electroretinogram (ERG) and the presence of abnormal accumulations of pigmentation in the mid-peripheral retina aid in the diagnosis. RP is inherited in an autosomal dominant, autosomal recessive, or X-linked fashion. The age of onset ranges from childhood to early adulthood. At least 36 different genetic loci have been associated with RP, and 27 mutated genes have been identified.

Rod degeneration is the primary pathological event in RP, resulting in the symptoms of night blindness and peripheral vision loss. The degeneration of the cones is secondary to the rod degeneration and results in complete loss of vision. Of the genes identified to date which cause retinal degeneration, many are involved in the phototransduction cascade. Any disruption of a component involved in phototransduction can lead to vision loss. For example, mutations have been identified in rhodopsin, the α- and β-subunits of phosphodiesterase, transducin, rhodopsin kinase, arrestin and guanylate cyclase. Disruption of the cascade at any of these levels results in the same RP disease phenotype. Mutations have also been found in structural proteins, mitochondrial genes, and transcription factors. The common and final element in all of these degenerative conditions is death of the photoreceptors through apoptosis (programmed cell death).

Retinal ischemia occurs when the blood supply is insufficient to support the metabolic needs of the retina. Many cases of visual loss and blindness are caused by diseases in which retinal ischemia is a central feature (e.g., diabetic retinopathy, retina vascular occlusion, and certain types of glaucoma). Neovascularization, which follows the ischemic event, is a common feature of many chronic eye diseases and is an important cause of blindness in the western world. Experimental models of retinal ischemia have been developed to elucidate the mechanisms involved. The most commonly used model is created collapsing the central retinal artery by increasing intraocular pressure (IOP) for one hour followed by reperfusion after reducing IOP to normal levels. A similar model involves ischemia-reperfusion through ligation of the optic nerve. Both models show that apoptosis is a key cause of the neuronal damage which results from the ischemic event. Neurons of the ganglion cell layer are the first to die, followed by those in the inner nuclear layer and then those in the outer nuclear layer. The involvement of caspase proteins, which are critical in apoptotic pathways, has been well documented.

Retinal detachment is a common form of retinal injury, and a significant cause of visual loss, especially if it involves the central macula. Recovery of vision depends on the nature and duration of the detachment. Obstacles to visual recovery involve changes in photoreceptor characteristics, such as shortening of the outer segments, retraction of rod terminals from the outer plexiform layer and opsin redistribution. In addition, there is remodelling of the synapses of second-order neurons and proliferation of retinal glial cells. Some of these changes are somewhat reversible, leading to gradual recovery of some vision after successful reattachment of the retina. However, the primary cause of visual loss is most likely the death of the photoreceptors, which occurs by the process of apoptosis and is irreversible.

Other diseases and disorders are also associated with inappropriate apoptosis. One such example is spinal muscular atrophy (SMA). In SMA, the primary cause of motor neuron death is the depletion of SMN protein. To date, SMN has been shown to be involved in several essential cellular processes, none of which have been directly linked to cellular attrition. However, motor neuron death clearly proceeds through apoptosis.

Apoptosis also plays an important role in the differentiation, development and homeostatic maintenance of skeletal muscle. The prevailing wisdom has been that fiber death in muscular dystrophy is typically necrotic. However, there is a growing evidence that apoptosis and necrosis are not always separable phenomena and that necrosis can be one outcome of an initially apoptotic process. For example, in the focal ischemia model of stroke using thread occlusion of the middle cerebral artery, established dogma states that the center of the infarct area dies by necrosis. Surprisingly, however, XIAP is dramatically protective in this model. Recent studies suggest the same situation is true in several muscular dystrophies. Markers of apoptosis and necrosis co-exist in the muscle of both Duchenne muscular dystrophy (DMD) patients and in the mdx mouse model for DMD. In the mdx mouse, an acute, necrotic degenerative phase begins at approximately three weeks postnatal and eventually resolves in adult mice. However, apoptotic myonuclei can be detected prior to this phase in otherwise normal fibers, peak at approximately four weeks when necrotic fiber death is obvious, and decline thereafter. Apoptosis is energy-dependent and studies have shown that a switch to the passive process of necrosis occurs when ATP is limited, a situation present in damaged DMD muscle fibers due to functional ischemia. Taken together, and by analogy with the focal ischemia model, this suggests that fiber death is initiated through apoptotic pathways but that necrotic degeneration quickly predominates.

XIAP is a member of the inhibitor of apoptosis (IAP) family of proteins which suppress programmed cell death by interacting with and inhibiting the catalytic activity of caspases. XIAP has been shown to confer resistance to apoptosis in a variety of cell death models. For example, using the four vessel occlusion (4-VO) model of forebrain ischemia, we have shown that virally mediated over-expression of XIAP prevents both the production of catalytically active caspase-3 and the degeneration of CA1 neurons in the hippocampus. Protection is seen at both the histological and functional levels, as assessed by immunohistochemistry for neuronal markers and by spatial learning performance in the Morris water maze. XIAP has also shown neuroprotection in an experimental model of Parkinson's disease. The administration of 6-hydroxy dopamine (6-OHDA) or 1-methyl-4-phenyl-1,2,3,4-tetrahydropyridine (MPTP) produces a Parkinson's disease-like syndrome in animals which manifests the neuropathologic and behavioural deficits observed in human disease. We and others have demonstrated that recombinant adenoviral vectors encoding XIAP, when injected into the striatum, protect dopaminergic neurons and promote functional rescue, as illustrated by rotational behaviour in rats. Furthermore, we have shown that MPTP mediated neurodegeneration of dopaminergic neurons in the substantia nigra is completely suppressed in XIAP transgenic mice, where the over-expression of XIAP is driven by the NSE (neuron specific enolase) promoter. Moreover, the brains of XIAP transgenic mice appear normal. XIAP over-expression has no apparent effect on neuronal cell counts in the hippocampus, substantia nigra or cortex in transgenic brains in comparison to wild-type brains. In the eye, intravitreal gene delivery of XIAP using an adeno-associated virus (AAV) shows protection of optic nerve axons in a hypertensive rat model of glaucoma and XIAP has been shown to protect axotomized retinal ganglion cells from cell death.

A number of studies have shown that gene delivery into the eye is an effective way of targeting and preventing the cell death that is associated with retinal degeneration. For example, wild type β-phosphosdiesterase (β-PDE) gene has been reintroduced into eyes of rd mice (which are deficient in β-PDE) using adenoviral and lentiviral vectors, and has shown a reduction in disease progression. An AAV carrying wild-type RPE65 has been used to restore vision in a canine model of childhood blindness. AAV delivery of ribozymes has been used to target (and degrade) mutant RNAs in a rat model of RP. These preclinical gene therapy trials have all targeted specific mutations; some trails have shown very dramatic results.

Gene therapy has shown promise in other diseases as well. Recent studies have demonstrated the introduction of lentiviral vectors expressing dystrophin into mouse models of muscular dystrophy (Bachrach et al., Proc. Nat. Acad. Sci. 101:3581-3586, 2004). Additional studies using herpes simplex virus 1, have expressed foreign genes in neurons in mice (Kennedy, Brain. 120:1245-1259, 1997). Successful experiments have also indicated that AAV2 based bcl-xl expression can protect cells from apoptosis in a model for ALS (Garrity-Moses et al., Muscle Nerve. 32:734-744, 2005).

Recent experiments have addressed the difficulties inherent in protein based therapies. Development of various protein transduction domain-protein fusions has demonstrated the feasibility of modifying proteins so that they can readily enter host cells. Fusions of XIAP to protein transduction domains (PTDs), such as the Drosophila homeotic transcription protein antennapedia (Antp), the herpes simplex virus structural protein VP22, and the human immunodeficiency virus 1 (HIV-1) transcriptional activator Tat protein have been used to protect host cells from apoptosis stemming from a variety of causes. These therapies allow for the transient introduction of XIAP into host cells.

Despite these advances, there is still a need for improved methods for treating retinal degeneration, muscular dystrophy, neurodegeneration, and other diseases and disorders associated with inappropriate apoptosis.

### SUMMARY OF THE INVENTION

The invention features the treatment of a degenerative disease in a patient with XIAP. In one aspect of the invention, XIAP is administered to the patient using the method of gene therapy. Specifically, the invention features a method of treating a degenerative disease in a patient in need thereof by administering an expression vector (e.g., an adeno-associated virus (AAV) or lentivirus) encoding XIAP (e.g., human XIAP). In this aspect of the invention the XIAP is positioned in the vector for expression in the desired tissue (e.g., retina, skeletal muscle, or motor neurons).

In another aspect, the invention features the method of treating a degenerative disease in a patient in need thereof by administering to the patient a protein preparation of XIAP (e.g., human XIAP). In this aspect of the invention, the protein preparation of XIAP includes a protein transduction domain-XIAP fusion. Examples of protein transduction domains suitable for this aspect of the invention include Tat, Antp, and VP22. In this aspect of the invention the protein preparation of XIAP is administered to the patient and enters the desired tissue (e.g. retina, skeletal muscle, or motor neurons).

The above methods of treatment may be used to treat retinal degeneration, wherein the retinal degeneration is a degeneration of photoreceptors, retinal ganglion cells, amacrine cells, bipolar cells, or horizontal cells. The above methods can be also be used to treat a patient diagnosed with retinitis pigmentosa, glaucoma, age-related macular degeneration, retinal detachment, or retinal ischemia. The invention features the above treatment delivered through intravenous, intraarterial, intraocular, intravitreal, subretinal, or transsceleral modes of injection.

Another feature of the invention is the use of the above treatment methods to treat a patient diagnosed with spinal muscular atrophy (SMA), spinobulbar muscular atrophy (SBMA), amyolateral sclerosis (ALS), muscular dystrophy, or skeletal muscle atrophy. Examples of muscular dystrophy are Duchenne muscular dystrophy, Becker muscular dystrophy, Emery-Dreifuss muscular dystrophy, limb girdle muscular dystrophy, myotonic dystrophy, oculopharyngeal muscular dystrophy, and distal muscular dystrophy. The invention features the above treatment delivered through intravenous, intraarterial, or intramuscular modes of injection.

In another aspect, the invention features the method of reducing cell death during transplantation of cells, the method including expressing XIAP in the cells and/or treating the cells with a protein preparation of XIAP, at a level and for a duration sufficient to reduce cell death during transplantation. The invention features the above methods, wherein the cells are selected from neural stem cells, muscle stem cells, satellite cells, liver stem cells, hematopoietic stem cells, bone marrow stromal cells, epidermal stem cells, embryonic stem cells, mesenchymal stem cells, umbilical cord stem cells, precursor cells, muscle precursor cells, myoblast, cardiomyoblast, neural precursor cells, glial precursor cells, neuronal precursor cells, hepatoblasts, neurons, oligodendrocytes, astrocytes, Schwann cells, skeletal muscle cells, cardiomyocytes, or hepatocytes.

Another aspect of the invention features a method of treating Parkinson's disease in a patient in need thereof, the method including administering cells capable of differentiating as dopaminergic neurons, the cells treated in any of the above conditions, into the patient under conditions that treat Parkinson's disease.

The invention also features a method of treating muscular dystrophy (e.g., Duchenne muscular dystrophy, Becker muscular dystrophy, Emery-Dreifuss muscular dystrophy, limb girdle muscular dystrophy, myotonic dystrophy, oculopharyngeal muscular dystrophy, and distal muscular dystrophy) or skeletal muscle atrophy in a patient in need thereof, the method including transplanting cells capable of differentiating as skeletal muscle cells, the cells treated in any of the above conditions, into the patient under conditions that treat the muscular dystrophy or skeletal muscle atrophy.

Another aspect of the invention features a method of treating cardiac injury in a patient in need thereof, the method including transplanting cardiomyocytes or cells capable of differentiating as cardiomyocytes, the cells treated in any of the above conditions, into the patient under conditions that treat the cardiac injury.

An additional feature of the invention is a method of treating liver disease (e.g., acute liver disease and chronic liver disease) in a patient in need thereof, the method including transplanting hepatocytes or cells capable of differentiation into hepatocytes, the transplanted cells treated in any of the above conditions, into the patient under conditions that treat the liver disease.

By “XIAP” is meant any polypeptide having the activity of full-length human XIAP protein. This activity is characterized by inhibition of apoptosis and/or binding caspase 3. Examples of XIAP includes full length XIAP, including human XIAP (e.g., genbank accession numbers aac50373, cab95312, aah32729, np_{—}001158, aaw62257, aac50518, aax29953, Q9R0I6, aah71665, and cai42584), and XIAP xenologues. Examples of XIAP xenologues are mouse XIAP (e.g., genbank accession numbers q60989 and np_{—}033818), rat XIAP (e.g., genbank accession numbers aag22969, aag41193, and aag41192), domestic cow (e.g., genbank accession numbers xp_{—}583068 and np_{—}001030370), zebrafish (e.g., genbank accession numbers np_{—}919377, aah55246, and xp_{—}689837), chimpanzee (e.g., genbank accession number xp_{—}529138), dog (e.g., genbank accession number abb03778), chicken (e.g., genbank accession number np_{—}989919), frog (e.g., genbank accession number np_{—}001025583 and bad98268), orangutan (e.g., genbank accession number cah91479), and catfish (e.g., genbank accession number aax35535).

The term “XIAP” also means any functional XIAP fragment, or any fusion of functional XIAP fragments. Examples of these fragments include those that consist of, consist essentially of, or include (i) BIRs 1-3, (ii) BIR3 and the RZF, (iii) BIR 3 (or a conformationally stabilized BIR of Ts-IAP, TIAP, hILP-2, or birc8), (iv) BIR2-3, (v) BIR2 and the RZF, (vi) BIR1-2, or (vii) BIR2 alone. Furthermore, “XIAP” embraces any of these fragments having an additional amino terminal methionine.

The term “XIAP” also means any fusion of full length XIAP, or a functional fragment thereof, with another polypeptide. These fusions include, but are not limited to, GST-XIAP, HA tagged XIAP, or Flag tagged XIAP. These additional polypeptides may be linked to the N-terminus and/or C-terminus of XIAP.

The term “XIAP” also includes any chimeric XIAP protein. By “chimeric XIAP” is meant a protein comprising a fusion of a XIAP domain or domains with a portion of another protein, wherein the chimeric XIAP retains the properties of human XIAP. Examples of chimeric XIAP proteins include the fusion of any of the above XIAP domains, or fragments thereof, to any domain or fragment of the following proteins: HIAP1, HIAP2, Livin, Survivin, Apollon, BRUCE, MLIAP, API1, API2, API3, API4, cIAP1, cIAP2, NAIP, MIHA, MIHB, MIHC, ILP, ILP-2, TIAP, TLAP, or KIAP.

The term “XIAP” is meant to include any protein with at least 70% sequence identity with human XIAP. The term also includes any conservative substitutions of amino-acid residues in XIAP. The term “conservative substitution” refers to replacement of an amino acid residue by a chemically similar residue, e.g., a hydrophobic residue for a separate hydrophobic residue, a charged residue for a separate charged residue, etc. Examples of conserved substitutions for non-polar R groups are alanine, valine, leucine, isoleucine, proline, methionine, phenylalanine, and tryptophan. Examples of substitutions for polar, but uncharged R groups are glycine, serine, threonine, cysteine, asparagine, or glutamine. Examples of substitutions for negatively charged R groups are aspartic acid or glutamic acid. Examples of substitutions for positively charged R groups are lysine, arginine, or histidine. Furthermore, the term XIAP includes conservative substitutions with non-natural amino-acids.

By “degenerative disease” is meant any disease characterized by abnormal apoptosis or necrosis and resulting loss of healthy tissue.

By “fusion” is meant the linkage of two molecules through a covalent bond.

By “treating” is meant administering or prescribing a composition for the treatment or prevention of a particular disease or disorder.

By “protein preparation of XIAP” is meant a preparation of XIAP capable of entering cells when administered to a patient.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “an amount sufficient” is meant the amount of a compound (e.g., an expression vector encoding XIAP or a protein preparation of XIAP) sufficient to treat or prevent a particular disease disorder in a clinically relevant manner. A sufficient amount of an active compound used to practice the present invention for therapeutic treatment of conditions caused by or contributing to a degenerative disease varies depending upon the manner of administration, the age, body weight, and general health of the patient. Ultimately, the prescribers will decide the appropriate amount and dosage regimen. Additionally, an effective amount may can be that amount of compound in the combination of the invention that is safe and efficacious in the treatment of a patient having the degenerative disease over each agent alone as determined and approved by a regulatory authority (such as the U.S. Food and Drug Administration).

### BRIEF DESCRIPTION OF THE DRAWINGS

FIGS. 1A and 1B are photographs showing a western blot confirming XIAP over-expression in the right eye of a rat given a subretinal injection of rAAV-XIAP. Evidence for the presence of the transgene is found in FIG. 1A, which was probed with an antibody to the hemagglutinin (HA) tag found on the transgene. The level of over-expression is shown in FIG. 1B, which was probed with an antibody to riap3, which reacts with endogenous rat IAP as well as the XIAP transgene. Arrows indicate the band of interest.

FIGS. 2A and 2B are photographs of retinal sections showing TUNEL staining at 24 hours post-MNU treatment. Note the fewer TUNEL positive cells (as shown by the dark pigment) in the XIAP-injected right eye (FIG. 2B) in comparison to the uninjected left eye (FIG. 2A). ONL, outer nuclear layer.

FIGS. 3A-3I are photographs of retinal sections at selected time points post-MNU injection. By 48 hours post-MNU, the XIAP-injected outer nuclear layer (FIG. 3C) has many more photoreceptor nuclei than the GFP-injected eye (FIG. 3A) or uninjected control (FIG. 3B). Differences are much more pronounced at 72 hours (compare FIG. 3F with FIG. 3D and FIG. 3E). At one week, the XIAP-injected eye shows preserved morphology of outer nuclear layer (FIG. 3I), but this layer has completely degenerated in GFP-injected (FIG. 3G) and uninjected controls (FIG. 3H).

FIG. 4 is a schematic illustration of representative fullfield flash ERGs from a control rat (two upper traces) prior to MNU injection. The lower two traces are ERGs from a XIAP-treated eye (OD) and untreated eye (OS) of an experimental animal at 7 days post MNU treatment. The intensity of the flash in all four traces is 0.25 cd.s/m². The XIAP-treated eye shows a recordable ERG with reduced a-wave and b-wave amplitudes. The unprotected eye shows an extinguished and nonrecordable ERG tracing.

FIG. 5 is a picture of a western blot confirming XIAP overexpression in the injected eye of a rat given a subretinal injection of rAAV-XIAP. This blot was probed with an antibody to the HA tag on the transgene, confirming the presence of the XIAP transgene in the retina.

FIG. 6 is a photomicrograph of retinal sections of XIAP treated eyes which show structural protection by XIAP in two transgenic rat models of retinitis pigmentosa. Subretinal injections of AAV XIAP were delivered into one eye of the rat, with the contralateral eye serving as a control. Note the multiple layers of photoreceptors in the ONL (see arrows) of the XIAP treated retina, in comparison to the thinner photoreceptor layer in the control eye the contralateral eye and in the GFP treated eye.

FIGS. 7A and 7B are graphs showing ONL thickness in XIAP treated eyes. XIAP treatment preserves the ONL of treated eyes in comparison to both uninjected control eyes (P<0.0001), and GFP injected eyes (P<0.005) for S334ter animals (FIG. 7A: XIAP N=16, GFP N=4). Similarly for P23H animals, XIAP treatment preserves the ONL of treated eyes in comparison to both uninjected control eyes (P<0.0001), and GFP injected eyes (P<0.0001) (FIG. 7B: XIAP N=15, GFP N=8). P<0.0001 using a one-tailed, paired t-test. Error bars represent S.E.M.

FIGS. 8A and 8B are photomicrographs showing that HA-immunofluorescence co-localizes XIAP protein to the area of neuroprotection (FIG. 8A). The contralateral, uninjected eye has a thinner outer nuclear layer (ONL) and shows no HA immunoreactivity, as expected (FIG. 8B).

FIG. 9A is a graph showing b-wave analysis of functional protection by AAV-XIAP in the P23H and S334ter RP lines up to 28 weeks after AAV injection. XIAP-injected eyes show significantly higher b-wave amplitudes (indicative of inner retinal function).

FIG. 9B is a graph showing a-wave amplitudes (indicative of photoreceptor function) in the P23H rats, but not in the S334ter line. Significance is seen at individual time points by Student t-test (stars on graph), and overall, using Anova FIG. 10A is a graph showing the comparison between ERG profiles of XIAP-treated and GFP-treated animals at 24 hours post-ischemia. XIAP-treated eyes have significantly higher b-wave amplitudes at most of the intensities of light tested.

FIG. 10B is a graph showing the comparison between ERG profiles of XIAP-treated and GFP-treated animals at 4 weeks post-ischemia. XIAP-treated eyes performed significantly better than GFP controls at the highest intensities of light.

FIGS. 11A-11F are photomicrographs showing retinal morphology at 24 hours and 4 weeks post-ischemia. Cross sections were stained with H&E. XIAP-treated retinas (FIGS. 11B and 11E) more closely resemble non-ischemic, untreated retinas (FIGS. 11A and 11D) up to 4 weeks post-ischemia. GFP-treated retinas (FIGS. 11C and 11F) show reduced thickness compared to non-ischemic, untreated samples as early as 24 hours post-ischemia. Fewer cells are present in GFP-treated retinas and greater disorganization of these cells is observed. GCL=ganglion cell membrane; IPL=inner plexiform layer; INL=inner nuclear layer; ONL=outer nuclear layer. Scale bar=65 μm.

FIGS. 12A and 12B are graphs showing neuronal counts of the inner nuclear layer (INL) in retinas treated with XIAP or GFP at T=0, 24 hours and 4 weeks post-ischemia. Cells were counted from haematoxylin and eosin stained cryosections taken through the para-papillary (FIG. 12A) and mid-periphery (FIG. 12B) region of retinas. Cell counts were pooled for untreated control retina. XIAP-treated retinas retained significantly more cells up to 4 weeks post-ischemia in the para-papillary region. * Represents p<0.05 using Mann-Whitney test.

FIGS. 13A and 13B are graphs showing the average retinal thickness following treatment with XIAP or GFP at T=0, 24 hours and 4 weeks post-ischemia. The INL in XIAP-treated eyes was significantly thicker than GFP controls up to 4 weeks post-ischemia in the parapapillary (FIG. 13A) and mid-peripheral (FIG. 13B) regions. * Represents p<0.05 using Mann-Whitney test. INL=inner nuclear layer.

FIGS. 14A-14C are photomicrographs showing an analysis of optic nerve cross-sections at 4 weeks post-ischemia. The cross-section of an ischemic, optic nerve from a XIAP-treated animal (FIG. 14B) had fewer of the characteristics associated with dying axons and looked similar to a normal eye (FIG. 14A). The ischemic eye (FIG. 14C) treated with GFP showed characteristics of dying axons including thickening of the myelin sheath (grey arrow) or increased glial tissue infiltration (black arrows).

FIG. 14D is a graph showing a significant decrease in axons was observed in GFP treated eyes. * Represents p<0.05 using Mann-Whitney test. OS=left, untreated retina; OD=right, treated retina. Scale bar=25 μm.

FIGS. 15A and 15B are photomicrographs showing a TUNEL assay in para-papillary region of rat retina at 24 hours post-ischemia. Dark staining indicates apoptotic cell bodies. Scale bar=50 μm FIG. 15C is a graph showing that there were significantly fewer TUNEL positive cells in XIAP-treated retinas compared to GFP-treated retinas. * Represents p<0.05 using Mann-Whitney test.

FIGS. 16A and 16B are photographs showing analysis of muscle from F1 offspring of mdx X UbcXIAP hemizygotes. In FIG. 16A, tail DNA was PCR amplified with XIAP-specific primers to identify mdx mice inheriting the UbcXIAP transgene. In FIG. 16B, tibialis anterior (TA) and diaphragm (Dia; mounted between liver slices) were haemotoxylin/eosin stained. Muscle in mdx mice transgenic for UbcXiap show reduced levels of cellular infiltration (arrowed) and fiber degeneration.

FIGS. 17A and 17B are illustrations showing that 24 h of serum starvation leads to atrophy and induces transient apoptotic protein expression in differentiated C2C12 myotubes. In FIG. 17A, C2C12 myoblasts were allowed to differentiate in 2% horse serum (HS) for 4 days, at which time fully differentiated myotubes were incubated in 2% HS (control) or serum-free media (starvation) for 24 hours. Images were taken to quantify mean myotube diameter on computerized software, and myotubes were scraped and lysed for protein and RNA collection. MURF-1 and Atrogin-1 mRNA were assayed using real-time RT-PCR. All data expressed as % change relative to control. In FIG. 17B, 5 day old myotubes were switched to serum-free media and protein samples collected at the times indicated, and western blots performed for the active fragments of caspase-3, -9, and PARP. β-actin was used as a control.

FIGS. 18A and 18B are illustrations showing that XIAP overexpression in C2C12 myotubes attenuates starvation-induced atrophy. C2C12 myoblasts were differentiated for 3 days. Adenovirus expressing XIAP (Ad-XIAP; MOI 5-50) were added to the media for 24 h, and protein samples were collected and western blotted for XIAP (FIG. 18A; arrowhead in upper panel). Adenovirus expressing GFP was used to examine transfection efficiency (FIG. 18A lower panel; Ad-GFP at MOI 50). 3-day-old C2C12 myotubes were incubated with Ad-XIAP (MOI 50), Ad-GFP (MOI 50), or no adenovirus for 24 h, followed by 24 h starvation in serum-free media. Preliminary measures of atrophy were examined (FIG. 18B). All data expressed relative to non-starved control cells.

FIGS. 19A-D are photomicrographs showing survival of mouse embryonic ventral mesencephalic xenografts that over-express XIAP under control of the ubiquitin promoter (ub-XIAP; FIGS. 19A and 19B) or wild-type (WT; FIGS. 19C and 19D) grafts from fetuses of the same mother 3 weeks after implantation into the 6-OHDA-denervated rat striatum. Recipients were not immunosuppressed. Note that a large TH-positive graft can be seen in FIG. 19A and that grafted ub-XIAP dopamine neurons appear healthy (arrows) with extensive fiber outgrowth (dashed arrows in FIG. 19B). By contrast, dopaminergic grafts from WT embryos appear to have been have been rejected (FIG. 19C), display pyknotic TH-positive neurons (arrows) and more limited fiber outgrowth (FIG. 19D). Boxed regions of FIGS. 19A and 19C correspond to areas shown in FIGS. 19B and 19D, respectively. Scale Bar=400 μm (FIGS. 19A and 19C); 100=μm (FIGS. 19B and 19D)

FIG. 20A is photograph showing that overexpression of XIAP has no gross morphological effects during development. Representative transgenic (Tg) and wild-type (wt) littermates are shown side by side.

FIG. 20B is a photograph of a western blot showing confirmation of transgene derived 6myc tagged human XIAP (65 kDa) and endogenous mouse XIAP (55 kDa) in skeletal muscle and heart muscle protein lysates.

FIG. 20C is a photomicrograph showing that myoblasts derived from UBC-XIAP transgenic mouse embryos display normal levels of fusion when exposed to reduced serum conditions. Top panel: phase contrast. Bottom panel: anti-MF20 immunoflourescence staining of total sarcomeric actin.

FIG. 20D is a graph showing that transgenic mouse derived myoblasts display increased resistance to apoptosis triggered by UV light exposure. Similar results were obtained following exposure to chemotherapeutic drugs (etoposide, camptothecin).

### DETAILED DESCRIPTION OF THE INVENTION

Aberrant apoptosis underlies many degenerative diseases. The invention features a method of treating patients with a degenerative disease with XIAP, a protein which blocks apoptosis. XIAP can either be administered through gene or protein therapy. A detailed description of the invention is recited below.

Gene Therapy

The invention features the method of gene therapy to express XIAP in a patient. In general, there are two approaches to gene therapy in humans. For in vivo gene therapy, a vector encoding the gene of interest can be administered directly to the patient. Alternatively, in ex vivo gene therapy, cells are removed from the patient and treated with a vector to express the gene of interest. In this method of gene therapy, the treated cells are then re-administered to the patient.

Numerous different methods for gene therapy are well known in the art. These methods include, but are not limited to, the use of DNA plasmid vectors as well as DNA and RNA viral vectors. In the present invention, these vectors are engineered to express XIAP when integrated into patient cells.

Adenoviruses are able to transfect a wide variety of cell types, including non-dividing cells. The invention includes the use of any one of more than 50 serotypes of adenoviruses that are known in the art, including the most commonly used serotypes for gene therapy: type 2 and type 5. In order to increase the efficacy of gene expression, and prevent the unintended spread of the virus, genetic modifications of adenoviruses have included the deletion of the E1 region, deletion of the E1 region along with deletion of either the E2 or E4 region, or deletion of the entire adenovirus genome except the cis-acting inverted terminal repeats and a packaging signal (Gardlik et al., Med Sci Monit. 11: RA110-121, 2005).

Retroviruses were among the first constructed human gene therapy vectors and, in general, are not able to transfect non-dividing cells. The invention includes use of any appropriate type of retrovirus that is known in the art, including, but not limited to, Moloney Murine Leukaemia Virus (MoMLV). The invention further embodies genetic modification of retroviruses including deletions of the gag, pol, or env genes. Using retrovirus constructs it is possible to target gene therapy vectors encoding for the expression of XIAP protein to specific tissues. This can be achieved through the fusion of part of the retrovirus env gene to a sequence encoding for the ligand to a tissue-specific receptor.

In another aspect, the invention features the methods of gene therapy that utilize a lentivirus vectors to express XIAP in a patient. Lentiviruses are a special group of retroviruses with the ability to infect both proliferating and quiescent cells. An exemplary lentivirus vector for use in gene therapy is the HIV-1 lentivirus. Previously constructed genetic modifications of lentiviruses include the deletion of all protein encoding genes except those of the gag, pol, and rev genes (Moreau-Gaudry et al., Blood. 98: 2664-2672, 2001).

Adeno-associated virus (AAV) vectors can achieve latent infection of a broad range of cell types, exhibiting the desired characteristic of persistent expression of a therapeutic gene in a patient. The invention includes the use of any appropriate type of adeno-associated virus known in the art including, but not limited to AAV1, AAV2, AAV3, AAV4, AAV5, and AAV6 (Lee et al., Biochem J. 387: 1-15, 2005). Previous experiments have shown that genetic modification of the AAV capsid protein can be achieved to direct infection towards a particular tissue type (Lieber, Nature Biotechnology. 21: 1011-1013, 2003).

Herpes simplex virus (HSV) replicates in epithelial cells, but is able to stay in a latent state in non-dividing cells. Under circumstances where it is desired to express XIAP in non-dividing cells, including neurons, the gene encoding XIAP can be inserted into the LAT region of HSV, which is expressed during latency. Other viruses that have been shown to be useful in gene therapy include parainfluenza viruses, poxviruses, and alphaviruses, including Semliki forest virus, Sinbis virus, and Venezuelan equine encephalitis virus (Kennedy, Brain. 120: 1245-1259, 1997).

Methods of gene therapy using cationic liposomes are also well known in the art. Exemplary cationic liposomes for use in this invention are DOTMA, DOPE, DOSPA, DOTAP, DC-Chol, Lipid GL-67™, and EDMPC. These chemicals can be used individually, or in combination, to transfect human cells with a plasmid constructed to express XIAP.

Another aspect of this invention is the methods of gene therapy that utilize DNA-polymer conjugates for the expression of XIAP in a patient. In this aspect of the invention, a vector constructed to express XIAP in a patient is combined with a polymer to achieve expression of XIAP without using a viral vector. Exemplary compounds for use in this embodiment of the invention are polyethyleneimine (PEI), polylysine, polylysine linked to nuclear localization signals, polyamidoamine, or polyarginine (Arg₁₆).

Another method of gene therapy uses a substantially purified DNA vector (naked DNA) for the expression of a protein of interest in a patient. This aspect of the invention features a treatment where the naked DNA is administered using an injection, a gene gun, or electroporation.

Another feature of this invention is the method of gene therapy where any of the above compositions are directed to certain tissue types. This feature of the invention includes the use of multi-valent antibodies to direct the above-described compositions to the desired tissue.

Using ex vivo gene therapy, an individual skilled in the art can be assured that XIAP protein will only be expressed in the desired tissue. In these applications, as well as applications where tissue specific expression of XIAP is not a concern, the above vectors can be constructed to constitutively express XIAP protein. Numerous constitutive regulator elements are well known in the art. Often, elements present in the native viruses described above are used to constitutively express a gene of interest. Other examples of constitutive regulatory elements are the chicken β-actin, EF1, EGR1, eIF4A1, FerH, FerL, GAPDH, GRP78, GRP94, HSP70, beta-Kin, ROSA, and ubiquitin B promoters.

For in vivo applications of gene therapy, the above vectors may be modified to include regulatory elements that confine the expression of XIAP to certain tissue types. Numerous examples of regulatory elements specific to certain tissue types are well known in the art. Of particular interest to the invention are elements that direct gene expression in the retina, nervous system, or muscle.

In order to treat retinal degeneration, it is desirable to specifically express XIAP protein in the retina. Several regulatory elements for use in gene therapy are well known in the art, including the mouse opsin promoter (MOPS) and ret-1 (a transcriptional element regulating rod opsin). Additional regulatory elements can be developed through the use of regulatory motifs common to many different retinal specific genes (Livesey et al., Current Biology. 10:301-310, 2000; Flannery et al., Proc. Natl. Acad. Sci. 94:6916-6921, 1997).

Also of interest are the methods of gene therapy directed towards the expression of XIAP specifically in muscle tissue. The use of the muscle creatine kinase regulatory element to direct tissue specific expression of transgenes is well known in the art. Additional muscle specific regulatory elements include the regulatory elements that control expression of myosin heavy chain, desmin, myoglobin, Pax 7, Pax 3, and alpha-actin.

An additional feature of this invention is the treatment of neuro-degenerative diseases with a vector expressing XIAP. Several regulatory elements are well known in the art to direct neuronal specific gene expression including the neural-specific enolase (NSE), and synapsin-1 promoters (Morelli et al. J. Gen. Virol. 80: 571-583, 1999).

In some embodiments of the invention it may be desirable to direct XIAP expression in an inducible fashion. Several methods of inducible transgene expression are widely used. These methods consist of the transfection of the patient's cells with multiple viral or plasmid vectors. Typically a first vector expresses the gene of interest under the control of a regulatory element that is responsive to the expression product of a second vector. The activity of this expression product is controlled by the addition of a pharmacological compound or some other exogenous stimulation. Examples of these systems are those that respond to tetracycline, mifepristone, ponasterone A, papamycin, tamoxifen, radiation, and heat shock (Robson et al., J. Biomed. Biotechnol. 2: 110-137, 2003).

Protein Therapy

Another feature of this invention is the method of treatment using a protein preparation of XIAP. In this method, XIAP protein is administered alone or is conjugated to some other factor that facilitates its translocation across the cell membrane and/or blood brain barrier. Briefly, protein therapy consists of forming fusion constructs with the protein of interest and a protein transduction domain (PTD). The three most commonly used PTDs are from the Drosophila homeotic transcription protein antennapedia (Antp), the herpes simplex virus structural protein VP22, and the human immunodeficiency virus 1 (HIV-1) transcriptional activator Tat protein (Wadia et al., Curr. Opin. Biotechnol. 13: 52-56, 2002). Additional studies have demonstrated the construction of modified PTDs which have enhanced translocation properties (Ho et al., Cancer Res. 61: 474-477, 2001). Recent reports have demonstrated the protective effect of administering TAT-XIAP fusion proteins in mice (Guegan et al., Neurobiol. of Dis. Published online Dec. 14, 2005) and Antp-XIAP in rats (Li et al., Experi. Neurol. 197:301-308, 2006; Fan et al., Neurochem. Int. 48:50-59, 2006).

Under certain circumstances it may be desirable to treat patients with XIAP through both protein and gene based therapy. Once integrated into the host cells, gene therapy will result in prolonged XIAP expression. In order to achieve therapeutically effective concentrations of XIAP immediately after the initiation of treatment, protein therapy can be administered in conjunction with the gene therapy. In certain circumstances, XIAP protein therapy alone (i.e., in the absence of XIAP gene therapy) may be employed.

Methods of Administration

In addition to intraarterial or intravenous injection, several methods for administering protein or gene therapy to a patient are well established. Possible methods of injection for retinal degeneration diseases include intraocular, intravitreal, subretinal and transsceleral modes of injection. For muscular degeneration diseases, intramuscular injection can be used, while intracerebral or intrathecal injection may be used for neurodegenerative diseases.

### EXAMPLE 1

### XIAP Rescue in a Chemotoxic Model of Retinal Degeneration

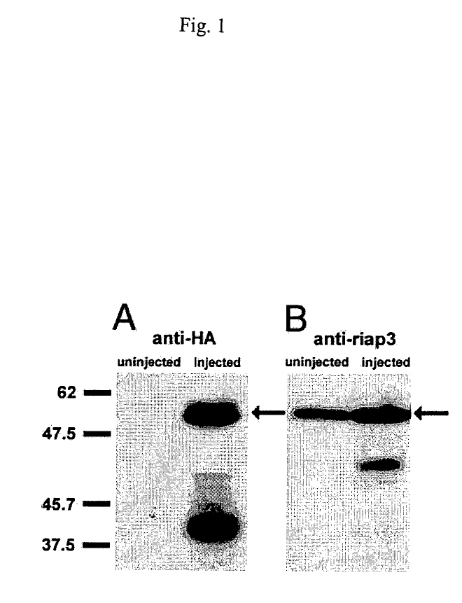

We tested the protective effects of XIAP in a chemotoxic model of retinal degeneration. This model involves the intraperitoneal injection of N-methyl-N-nitrosourea (MNU) in rats, which results in massive photoreceptor cell apoptosis by 24 hours, as reflected by TUNEL staining in the outer nuclear layer. By 72 hours post-MNU injection, there is a complete destruction of the outer nuclear layer of the retina. We used serotype-2 AAV vectors to introduce XIAP into the rat eye. Serotype-2 AAV transgene expression begins at approximately two weeks post-delivery and appears to be optimal at 6 weeks post-delivery. We performed subretinal injections of AAV encoding XIAP or green fluorescent protein (GFP-control) driven by the mouse opsin promoter (MOPS). The right eye of the animal was given the virus and the left eye was used as a normal, uninjected control. Six weeks post-injection, we delivered an intraperitoneal injection of MNU at a dosage of 60 mg/kg body weight and we followed photoreceptor fate for one week. XIAP over-expression was confirmed at 6 weeks post injection using western blot analysis (FIGS. 1A and 1B).

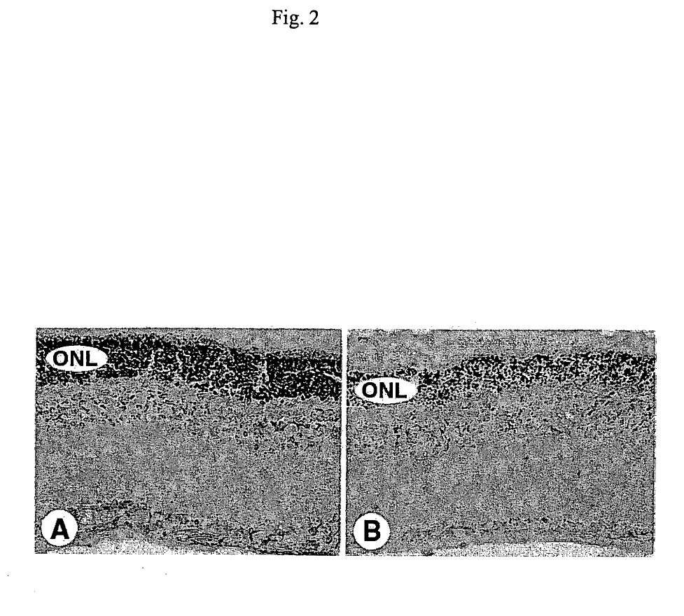

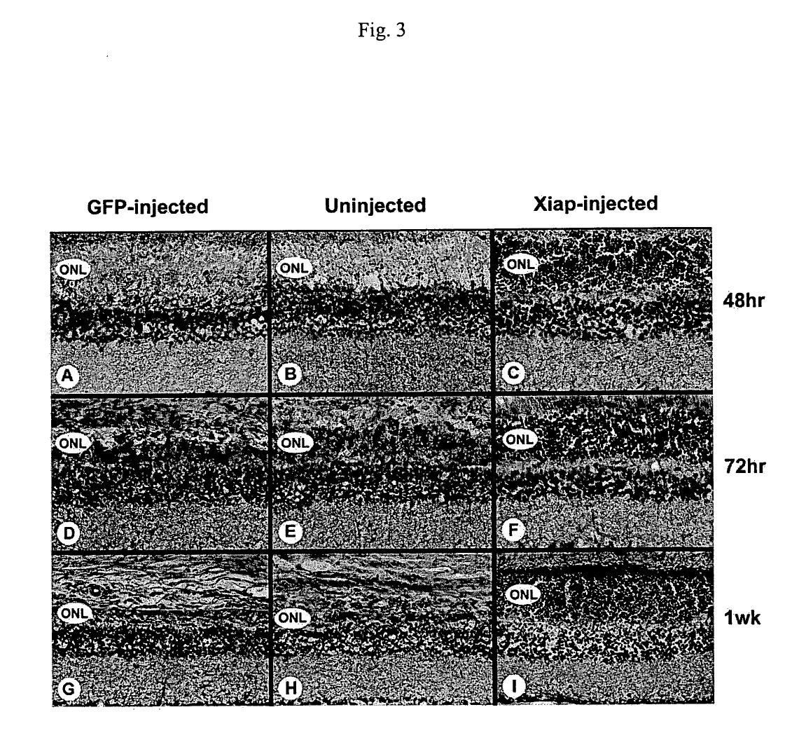

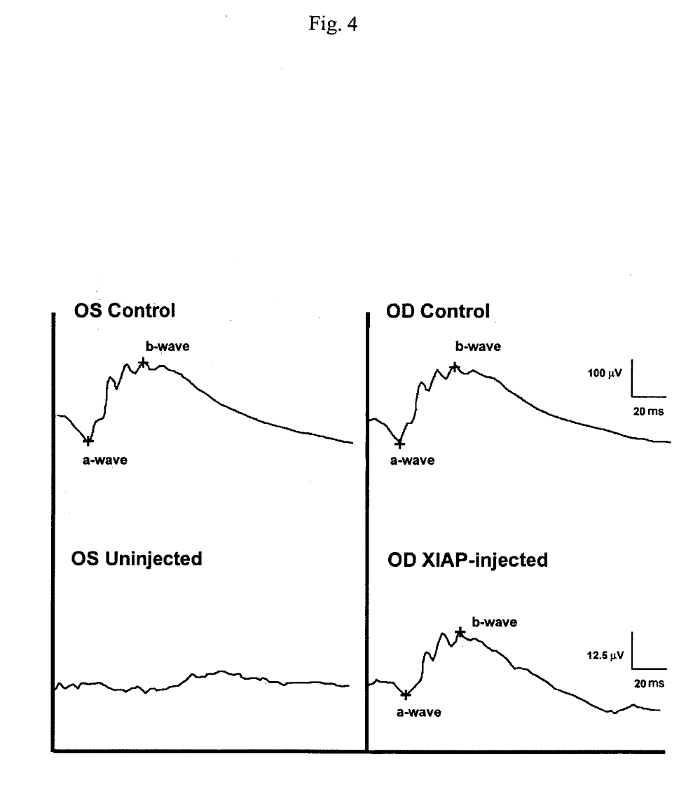

XIAP protein expression conferred significant protection to the photoreceptor layer of the retina. No such protection was seen in GFP-injected eyes and uninjected controls. The XIAP protective effect was first apparent at 24 hours post-MNU, where a much larger number of cells were undergoing apoptosis in the left uninjected eye (and in the GFP injected eye) relative to the XIAP injected right eye (FIGS. 2A and 2B). Histological examination of MNU treated eyes showed visible changes at 48 hours, and by 72 hours post MNU, the ONL of the retina was virtually obliterated in the uninjected or the GFP-injected eyes. However, eyes receiving XIAP showed a large degree of histological protection in the region of the injection up to 1 week post-MNU (FIG. 3), at which time the experiment was terminated. Moreover, a good proportion of these eyes (2/6) also showed functional protection, as evidenced by electroretinography (ERG; FIG. 4).

The present study with XIAP represents the first evidence of functional protection of photoreceptors following MNU treatment. The degree of functional protection is somewhat limited and there are two possible explanations for this. Firstly, the 2 μl injection of the virus covers at best 10-20% of the rat retina; consequently, there are adjacent treated and untreated areas. The full field ERG records the response of the whole retina. Thus, any localized electrical response which results from neuroprotection would be somewhat diminished by the majority of the retina which was not covered by the injection and is undergoing cell death. Clinically, this can be addressed by modifying the administration regime. Secondly, MNU is a potent alkylating agent and is expected to cause severe damage to the DNA even before the cell initiates apoptotic signals. It is thus quite remarkable that functional protection was observed at all, and this result suggests that XIAP may have additional protective effects upstream of its role in caspase inhibition.

Given that XIAP shows such potent protective effects in the MNU model, which is one of the most severe retinal degeneration models available, one would expect that XIAP would be an ideal gene therapeutic agent for less severe and much more slowly progressing forms of retinal degeneration which are characteristic of human disease. Furthermore, since XIAP has shown neuroprotective effects in the 4-VO model of brain ischemia, we predict that XIAP will protect against cell death during retinal ischemia.

### EXAMPLE 2

### XIAP Rescue in a Rhodopsin Transgenic Rat, a Genetic Model of Retinitis Pigmentosa

The P23H transgenic rat has a mutation (histidine substituted for proline in position 23) in the rhodopsin gene. The same mutation is responsible for the most common form of autosomal dominant retinitis pigmentosa (ADRP) in North America. Twelve percent of all human ADRP cases are caused by this mutation, and thus, any therapeutic benefits by XIAP that are seen in this animal model could be directly applicable to the human disease.

There are several lines of P23H transgenics available, and these differ in the rates of retinal degeneration. For all of them, photoreceptor loss begins at approximately 2 weeks of age, but the speed of progression of the disease is quite variable in the different lines. Line 1 has a relatively rapid retinal degeneration such that by 3 months, only a few layers of photoreceptors remain (10-12 layers are present in the normal retina). The S334ter transgenic rat (line 4) contains a truncated rhodopsin transgene and shows a similar rate of retinal degeneration to P23H line 1.

XIAP rescue in these rat models of retinitis pigmentosa is examined using the following methods.

Methods

Injection Methods

Serotype-5 AAV carrying XIAP under the control of the chicken beta-actin promoter (AAV-CBA-XIAP) and control virus encoding GFP (AAV-CBA-GFP) have viral titres of approximately 1×10¹⁴ viral particles/ml. Serotype-5 AAV has high affinity for photoreceptors, and expression from the transgene is optimal within one week of delivery, ensuring that the gene therapy is effective early in the progression of the disease. XIAP constructs are tagged with haemagglutinin (HA) to allow monitoring of the transgene. Rats are anaesthetized by isofluorane inhalation at post-natal day 15 (when the eyes open), and a small hole is made through the inferior cornea with a 28-gauge needle. A blunt 33-gauge needle is inserted through the hole, manoeuvred around the lens and through the retina, and 2 μl of AAV-XIAP is delivered into the subretinal space. Control injections are made with AAV-GFP to test the effects of the virus and injection procedure. Fluorescein (1% v/v) in the injection solution allows the visualization of the injection site using a dissecting microscope, making it easy to determine whether a successful subretinal injection has been delivered. Right eyes are injected in each animal, and left eyes serve as controls. This eliminates any inter-animal variation in ERG profiles. In our hands, subretinal injection causes a localized retinal detachment which is accompanied by a reduction in ERG b-wave amplitude. Full recovery of ERG amplitude occurs within 3-4 weeks, which is an indication that the retinal detachment and any associated damage has resolved. For each of the P23H and the S334ter lines, the right eye of 45 animals are subretinally injected at post-natal day 15 with AAV-XIAP or AAV-GFP. The left eye serves as a non-injection control. The ERGs are recorded and further sampled every month (i.e., at 1, 2, 3, 4 and 5 months of age). Animals are sampled at various time points for protein analysis and for histology (see histological analysis below for details).

ERGs

The functional analysis of the retina is performed using both full-field and multi-focal ERGs. The full-field ERGs record the response of the whole retina, while multifocal ERGs permit the simultaneous assessment of retinal function in multiple quadrants of the retina.

Full-field ERGs: Full-field scotopic/photopic ERGs are generated using the ESPION system (Diagnosys LLC, Littleton). Animals are dark-adapted overnight. Under ketamine/xylazine anaesthesia, small platinum electrodes are placed on both corneas following pupil dilation and a drop of 2.5% methylcellulose is used to maintain corneal hydration. Reference and ground electrodes are placed in the mouth and tail, respectively. Single flash stimuli (4 msec duration) are presented at intensities ranging from 0.001cd.s/m² to 25 cd.s/m² and the a- and b-wave responses are measured. In simplified terms, the a-wave is the initial negative potential seen as a result of hyperpolarization of the photoreceptors. The b-wave amplitude is the positive potential measured from the trough of the a-wave to the highest point in the trace and represents the potential generated by cells in the inner nuclear layer. The implicit times and amplitudes of the a-wave and b-wave are derived through visual inspection. The a-wave and b-wave amplitudes are plotted against increasing stimulus luminance. The increase in the amplitude of the b-wave is described empirically by the Naka-Rushton equation: R/Rₘₐₓ=Lⁿ/(Lⁿ+Kⁿ). The parameters Rₘₐₓ, n and K are considered independent measures of retinal responsiveness, retinal homogeneity, and retinal sensitivity, respectively.

Multifocal Electroretinograms (mERG): In our models, subretinal injections of XIAP are applied to one eye and the full field ERG is used to compare the resultant electroretinal function between treated and untreated eyes. The subretinal injection of XIAP may not be distributed to the entire retina of the treated eye; consequently, there may be adjacent areas of treated and untreated retina. Multifocal ERG allows objective measurement of electroretinal function of treated and untreated regions in the same eye. The mERG allows for the determination of the exact coverage of a subretinal injection, and whether there is partial or full protection at the site of injection. This helps to determine whether the virus is infecting and protecting all cells at the injection site, or just a subset of cells. mERGs are recorded in anesthetized animals following pupil dilation, using small platinum electrodes placed on the cornea with constant maintenance of corneal hydration. Reference and ground electrodes are placed in the mouth and tail, respectively. A 19 hexagonal stimulus array is presented under direct observation through a fundus/stimulator system (VERIS Version 5.0, Electro-Diagnostic Imaging Inc, San Mateo, Calif.). The stimulus is centered on each animal individually such that regions of XIAP-protected retina and unprotected retina are sampled simultaneously. The stimulus is presented with one stimulus field followed by nine blank fields at a presentation rate of approximately ten presentations per second. The luminance of bright hexagons is maintained at 80 cd/m² and the dark hexagons are presented at 1 cd/m². An m-12 sequence is used to elicit approximately 4,096 stimulus presentations. The N1, P1, and N2 components are analyzed with regards to response latency and amplitude.

Histological Analysis

Representative animals are sacrificed post-ERG at the various time points discussed above and XIAP neuroprotection assayed. In a small subset of animals, western blots are generated with protein extracts from right (XIAP-injected) and left (control) eyes. These blots are probed with antibodies to the HA tag (which should only hybridize to injected eyes) and with anti-RIAP3, a rabbit polyclonal antibody to rat XIAP which we have generated. RIAP3 staining reveals the degree of over-expression of the transgene in relation to endogenous levels of IAP. Some animals undergo transcardiac perfusion with 4% formaldehyde, and eyes scored, removed and embedded for cryosectioning. Scoring allows the orientation of the eye in the embedding process and helps in the approximate localization of the injection (all injections are done in the naso-inferior quadrant of the eye). In tissue sections, we detect the transgene immunohistochemically using an anti-HA antibody. A large number of sections are examined with this antibody to determine the extent of coverage of the injection. Intervening sections are analyzed using TUNEL to detect apoptotic cells. The percentage of TUNEL positive cells in the various layers of the retina is compared between XIAP-treated and untreated eyes. Some sections are processed for hematoxylin/eosin staining and cell counts performed in the various cell layers to assess the degree of neuroprotection.

Results

Homozygous P23H breeding pairs were received from Matt La Vail, (UCSF). Homozygous matings were set up for maintaining the colony, and homozygous males were bred to wild-type Sprague Dawley females for the gene therapy experiments. Multiple heterozygous litters were obtained, and the rat pups were injected with the XIAP and GFP constructs as outlined above. We tested serotype-2 MOPS-XIAP and MOPS-GFP AAV constructs, as well as serotype 5 virus where XIAP and GFP are driven by the chicken β-actin (CBA) promoter.

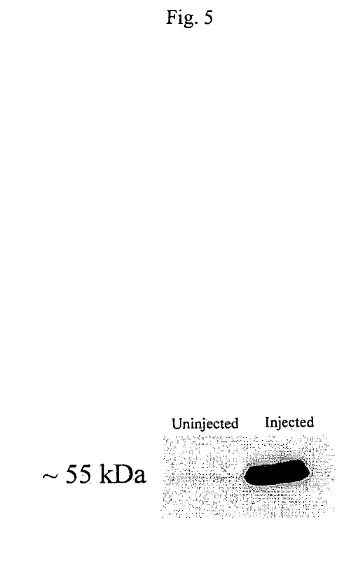

Western blot analysis confirmed that the exogenous XIAP is overexpressed at the protein level in the retinas of the injected eyes at approximately two weeks post injection (FIG. 5). The XIAP injected retinas show high levels of HA-tagged protein, which is absent in the contralateral control eyes. Given that the protein extract was made from the whole retina, and only a fraction of the retina was covered by the subretinal injection, the level of XIAP overexpression at the site of injection would be even greater than the western blot indicates. The level of transgene expression was examined in several eyes and was quite variable, suggesting that different XIAP treated eyes may have differing levels of protection depending on the level of XIAP overexpression.

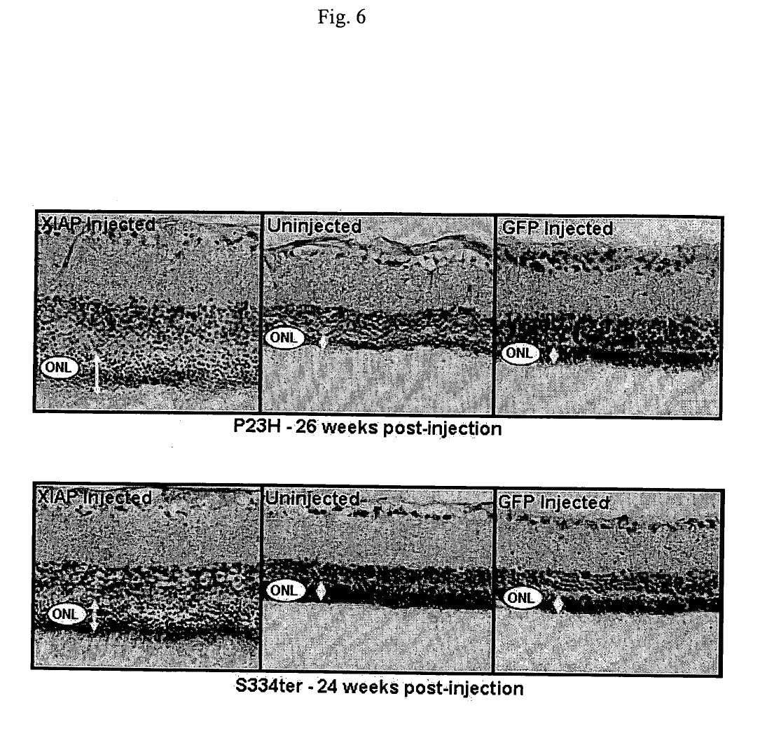

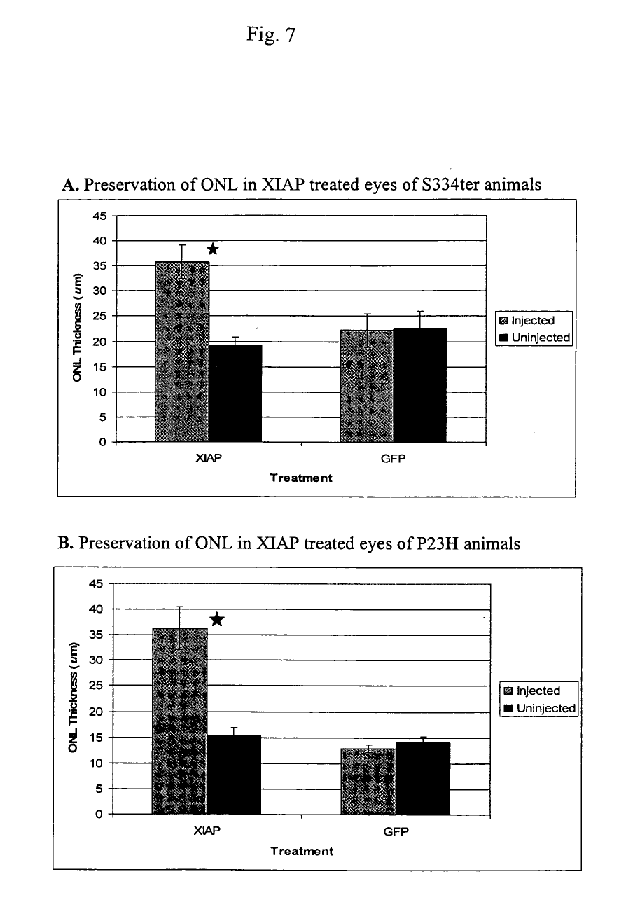

All animals were sampled between 24 and 32 weeks post injection. A high degree of structural protection was evident in XIAP treated eyes of both P23H and S334Ter animals up to 32 weeks post-injection, relative to the contralateral untreated control eye and also a similarly age-matched GFP injected control eye (FIG. 6). Measurements of ONL thickness in these animals revealed a highly significant preservation of ONL morphology in XIAP treated eyes relative to both uninjected and GFP injected control eyes (FIGS. 7A and 7B).

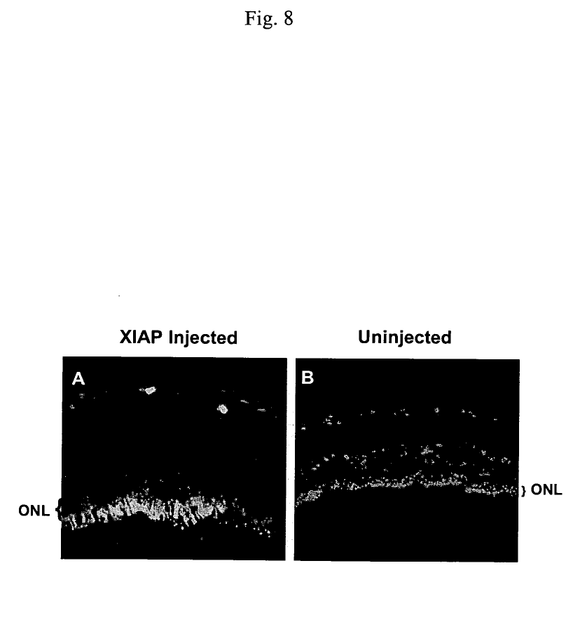

Immunohistochemistry was used to confirm the coincidence of XIAP overexpression in the areas of the retina showing preservation of photoreceptor cells (FIGS. 8A and 8B). Using HA immunofluorescence, expression of HA-XIAP is clearly seen in photoreceptor inner segments and to a lesser extent in the ONL (FIG. 8A). These same sections were also treated with a DAPI nuclear stain in order to correlate the XIAP expression with ONL thickness. These DAPI stained images were overlayed with HA fluorescence images, which clearly demonstrates the area with preserved ONL morphology coinciding perfectly with the area where the exogenous XIAP is expressed. As expected, the retina of the uninjected eye shows no HA immunofluorescence and a uniformly diminished ONL (FIG. 8B).

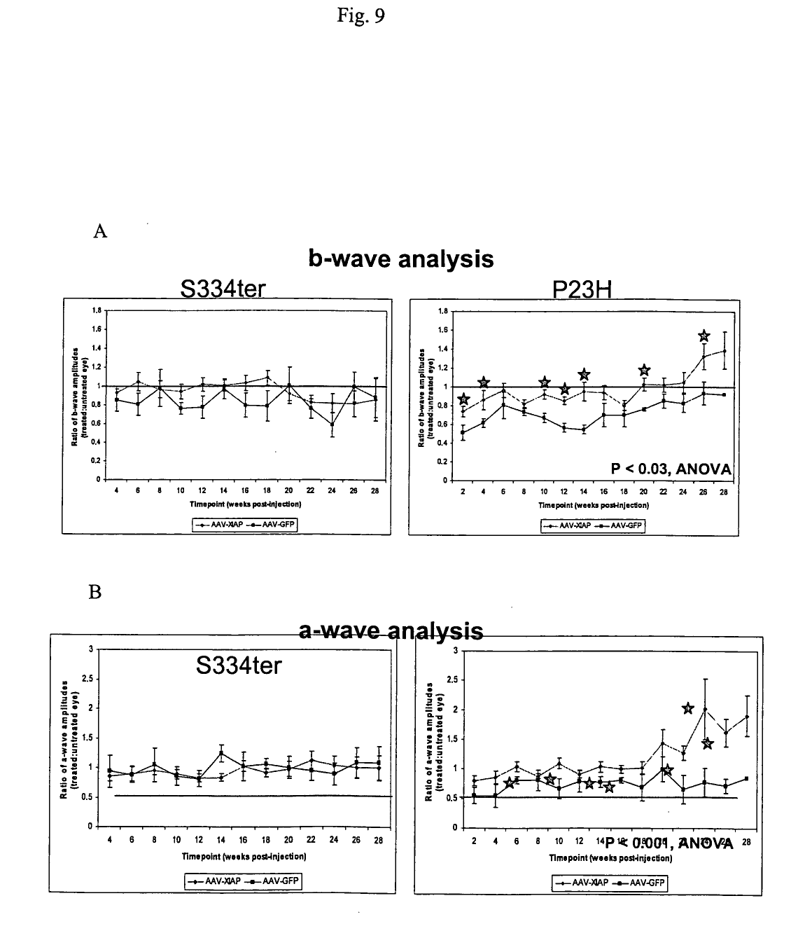

Ratios of the a- and b-wave amplitude in the treated eye to that in the untreated eye were used to detect preservation of function in XIAP treated eyes. The b-wave is a reliable measure of overall retinal function, and the a-wave is generated more specifically by the photoreceptor cells. For the S334ter group, up to approximately 18 weeks post-injection the b-wave amplitude ratios for the XIAP treated animals appeared to be somewhat, although not significantly, better than the GFP control ratios (FIGS. 9A and 9B). After 18 weeks post-injection, the performance in the XIAP group declined and the ratios were comparable to those for the GFP control group. Overall, there was no lasting or significant functional protection resulting from XIAP treatment in the S334Ter animals as assessed by a-wave or b-wave ratios.

For the P23H group, a-wave and b-wave ratios revealed clear functional protection in XIAP-treated retinas (FIGS. 9A and 9B). By 20 weeks post-injection the a-wave and b-wave ratios for the XIAP group crossed 1 and continued to increase up to the final timepoint of 28 weeks post-injection. This indicated that not only were the XIAP treated eyes doing better than the GFP treated control eyes, they were also doing better than the uninjected control eyes that had no surgical manipulation. To test for an overall effect, animals included in the study up to 26 weeks post-injection (XIAP N=12, GFP N=3) were examined and repeated measures ANOVA with Bonferroni-Dunn correction revealed a significant difference between the XIAP and GFP treated groups (P<0.03).

### EXAMPLE 3

### XIAP Protection of Different Layers of the Retina Against Retinal Ischemia

A number of studies have shown that cell death by apoptosis is a key event in retinal ischemia. Cell death occurs initially in the ganglion cell layer and this is followed by apoptosis of the cells in the inner and outer nuclear layers. The involvement of caspases has been well documented and inhibitors of caspases have been shown to be neuroprotective. Since XIAP is a potent inhibitor of caspases 3, 7 and 9 and has been shown to provide neuroprotection in a number of neurodegenerative models, including the 4-vessel occlusion model of neuronal ischemia, we predict that XIAP should have a similar neuroprotective effect in retinal ischemia.

Methods

AAVs encoding HA-tagged XIAP or GFP driven by the chicken beta actin (CBA) promoter were generated in the laboratory of Dr. William Hauswirth (University of Florida, Gainesville). Following anaesthesia and pupil dilation, 2 μl of CBA-XIAP or CBA-GFP virus is delivered into the vitreous of the right eye of Sprague Dawley rats. The CBA promoter is used in the intravitreal delivery of the virus because this promoter expresses in all cell types. Since the intravitreal injection will target ganglion cells as well as cells in the inner nuclear layer (bipolar, amacrine and horizontal cells) a universal promoter is required to ensure expression of the transgene in the diverse cell types. At six weeks post-injection, retinal ischemia is induced. Room temperature is kept at 24° C., and corneal temperature is measured. Preliminary experiments have revealed that room temperature and corneal temperature are crucial parameters in the delivery of a proper ischemia, and that temperature fluctuations of as little at 1° C. can have large effects on neuroprotection. Following anaesthesia, the anterior chamber of the right eye is cannulated with a 30-gauge needle connected to a saline reservoir and a manometer to monitor intraocular pressure. Intraocular pressure is raised to 110 mm Hg by raising the saline reservoir for 60 minutes. This increase in pressure collapses the central retinal artery. Retinal ischemia is confirmed by whitening of the iris and loss of red reflex, which are easily seen in the non-pigmented eye of the Sprague Dawley rats. After 60 minutes of ischemia, the intraocular pressure is normalized and the needle withdrawn. ERG analyses is conducted pre-ischemia, and at 24 hrs, 2 weeks and 4 weeks post-ischemia. Animals are sampled at time 0 (6 animals), 24 hrs (6 animals), and 4 weeks (12 animals) post-ischemia. Time 0 eyes are used to confirm the presence of the transgene through western blot analysis and immunohistochemistry with anti-XIAP, anti-HA or anti-GFP antibodies. Animals from all the time points (except the ones to be used for western analysis) undergo transcardiac perfusion with 4% formaldehyde, and eyes are removed and embedded for cryosectioning. Sectioned eyes are processed for TUNEL analysis, immunohistochemistry, or basic histology as outlined above in Example 2. Portions of the optic nerve are sampled in the 4-week group, embedded in Epon and sectioned at 0.5 μm. Axon counts are conducted following staining with 1% toluidine blue. The 4-week time point is used for this analysis because by this time, apoptosing axons have fully degenerated. Cell counts are conducted in the ganglion cell layer and the inner nuclear layer (INL), which are the layers most severely affected by the ischemia.

Results

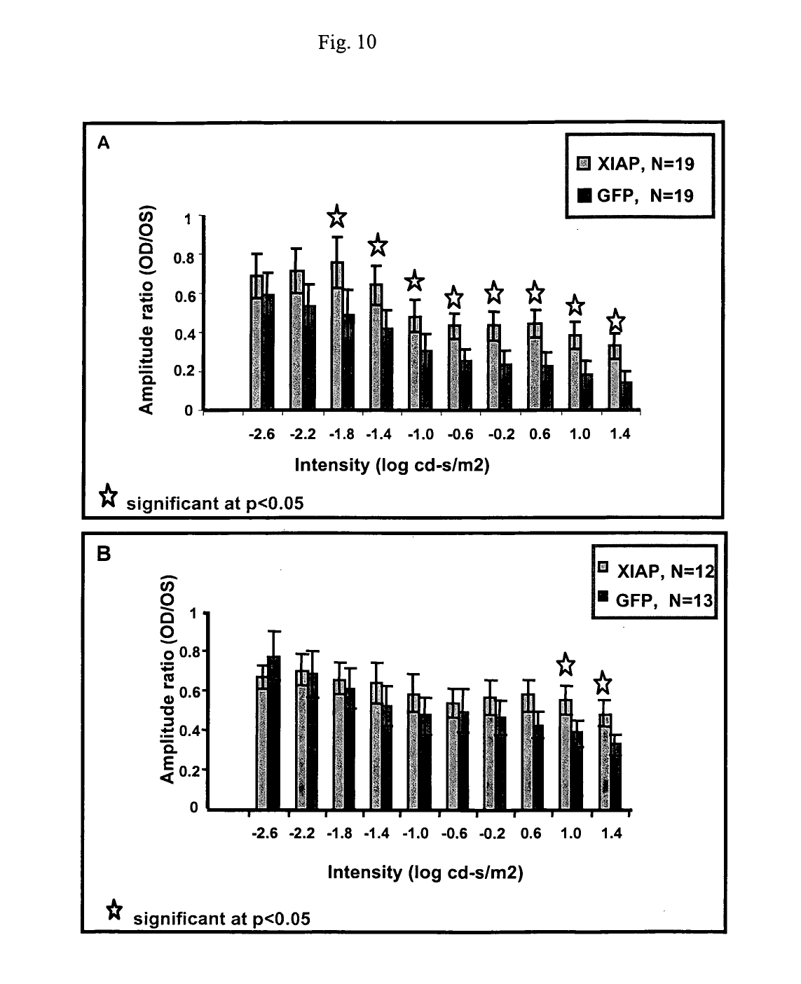

Ischemia was induced in the right eye in rats by increasing intraocular pressure and collapsing the central artery. Left eyes served as non-ischemic, untreated controls. Scotopic-photopic electroretinography (ERG) was performed bilaterally on rats in both the XIAP and GFP groups 24 hours post-ischemia, and 4 weeks post-ischemia. ERG waveform morphologies of rat ischemic eyes were characteristic of those found in humans. A hyperpolarizing a-wave was present as expected, but there was no recovery of the b-wave above baseline levels in untreated, ischemic eyes. This is the classic negative ERG characteristic of central retinal arterial occlusion. To assess b-wave amplitude preservation, the b-wave amplitude of the treated eye (OD) was divided by that of the contralateral, untreated eye (OS) to give a b-wave amplitude ratio (OD:OS). The closer the ratio was to 1.0, the better the preservation of the b-wave amplitude. At 24 hours post-ischemia, we observed that the XIAP-treated eyes possessed b-wave amplitude ratios that were closer to 1.0 in comparison to the GFP-treated eyes (FIG. 10A). The two groups were significantly different for all except the lowest intensities. At 4 weeks post-ischemia, both groups showed better, but not full recovery. The b-wave amplitude ratio in the XIAP-treated group was significantly higher than that of the GFP-treated group at the two highest light intensities (FIG. 10B).

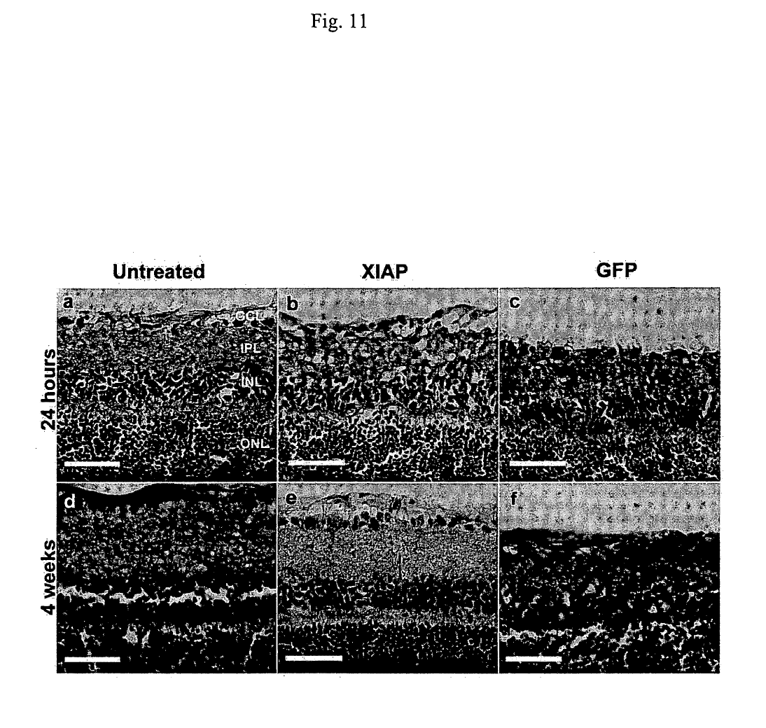

The structural effects of ischemia were analyzed on images of retinas that had been stained with haematoxylin and eosin. At 24 hours post-ischemia, there was no evident effect in XIAP-treated ischemic retinas in comparison to non-ischemic, untreated retinas (FIGS. 11A-11D). In GFP-treated ischemic retinas, the entire retina was reduced in thickness in comparison to non-ischemic, untreated retinas by 24 hours (FIG. 11C). Inner retinal loss in GFP-treated retinas at 4 weeks post-ischemia was more pronounced with greater disorganization of cells and thinning of retinal layers (FIG. 11F). Retinas treated with XIAP showed better preservation even at 4 weeks post-ischemia (FIG. 11E).

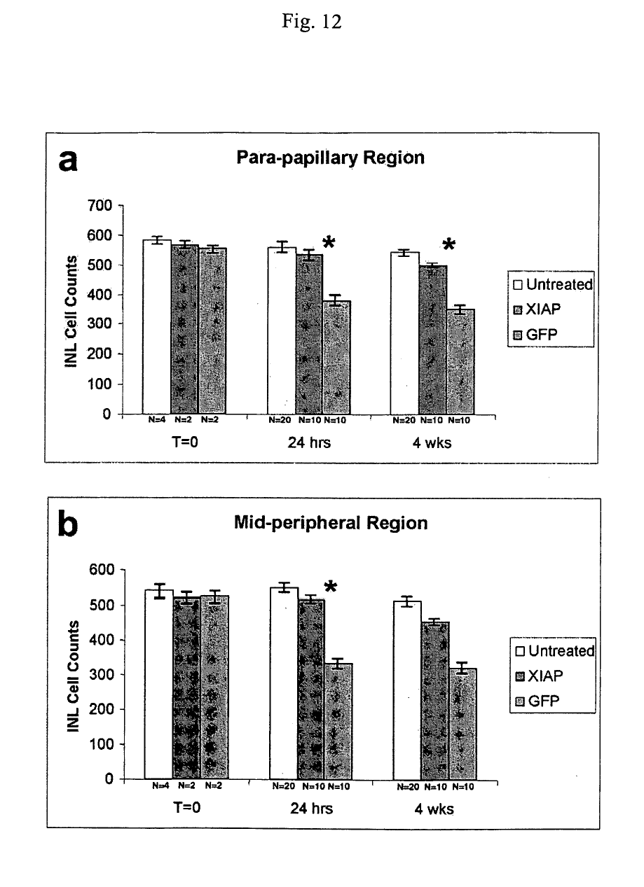

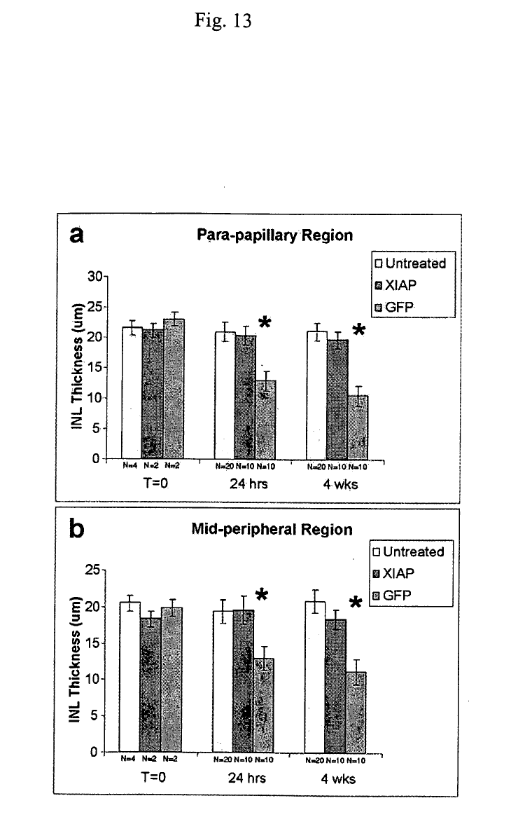

Measurements of the various retinal layers confirmed our histological observations. Cells in the INL were counted and means for the para-papillary and mid-peripheral retina were determined (FIGS. 12A and 12B). When compared to rats at T=0 and non-ischemic, untreated controls, eyes injected with AAV-XIAP at 24 hours post-ischemia showed little sign of retinal loss at the para-papillary (FIG. 12A) and mid-peripheral regions (FIG. 12B; p=0.01). Loss of retinal cells in XIAP-treated retinas was somewhat more evident at 4 weeks post-ischemia (FIGS. 12A and 12B). However, the number of cells remaining in the INL of XIAP-treated eyes at the para-papillary region was significant compared to GFP-treated eyes (FIG. 12A; p=0.03). Measurements of the thickness of the INL confirmed our cell counts. Treatment with XIAP significantly preserved INL thickness at the para-papillary and mid-peripheral regions of the retina at 24 hours and 4 weeks post-ischemia (FIGS. 13A and 13B; p=0.04 and p=0.02, respectively) in comparison to GFP-treatment.

Optic Nerve Analysis

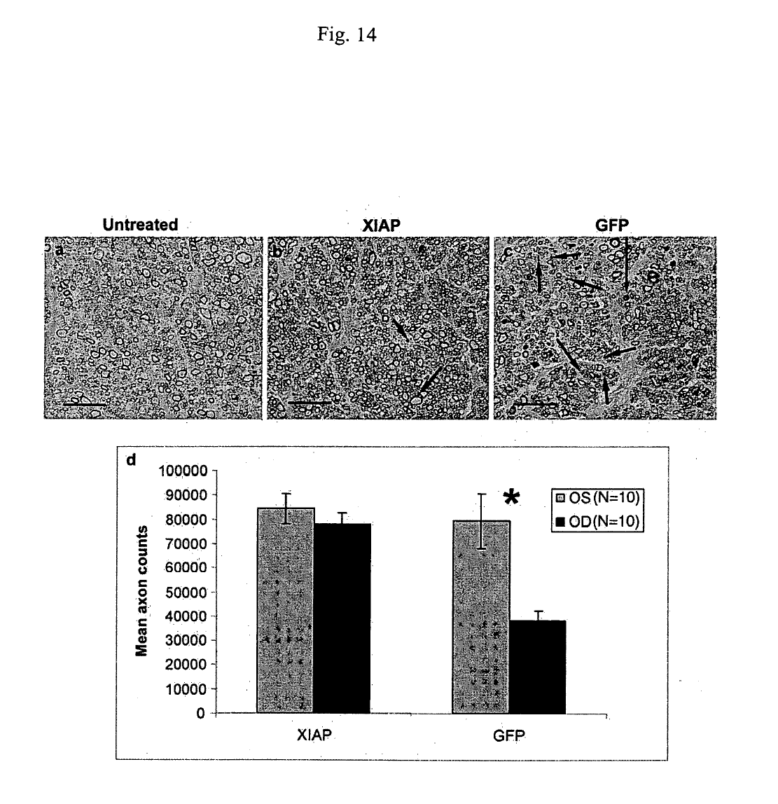

The effect of cell loss from the inner retina was analyzed in cross-sections of optic nerves. A marked reduction in size and a change in shape were detected in the GFP-treated samples relative to the untreated and XIAP-treated samples (FIGS. 14A-14C). We observed many dying axons in GFP-treated samples, characterized by an unhealthy myelin sheath (FIG. 14C; grey arrow). In addition, some dying axons exhibited moderate to heavy discolouration. We also observed greater infiltration of glial tissue in GFP-treated samples compared to XIAP-treated samples (FIGS. 14B and 14C; black arrows). There was no significant difference in the number of axons in XIAP-treated eyes compared to non-ischemic, untreated controls (FIG. 14D). However, a significant reduction in axonal counts was observed in GFP-treated eyes compared to controls (FIG. 14D; p=0.001).

TUNEL Analysis

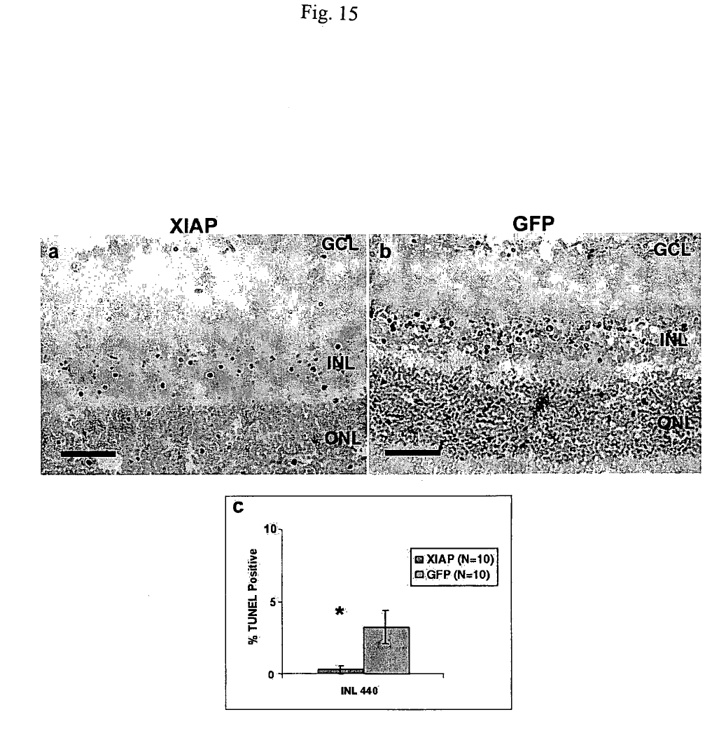

To verify that cell loss was due to apoptosis, TUNEL analysis was performed. At 24 hours post-ischemia, more TUNEL-positive staining was noted within the INL and GCL cells in GFP-treated retinas (FIG. 15B) compared to the XIAP-treated group (FIG. 15A) in both the para-papillary and mid-peripheral regions. There was a significant decrease in the percentage of TUNEL-positive cells in the para-papillary region of the INL in XIAP-treated retinas compared to the GFP-treated retinas (FIG. 15C; p<0.05).

These data demonstrate that a single intravitreal injection of rAAV-XIAP is capable of delivering significant neuroprotection to the retina. Furthermore, we observed significant preservation of not only the ganglion cells where our constructs are primarily expressed but also the INL and IPL. Neuroprotection of the ganglion cells by XIAP also results in preservation of the inner retina, since INL neurons synapse with ganglion cells and will consequently be affected by their survival. The persistence of the neuroprotective effects at 4 weeks following ischemia suggests XIAP confers permanent protection. Thus, XIAP-mediated gene therapy holds promise as a therapy in the clinic.

It is important to note, however, that gene therapy in retinal ischemia using AAV vectors provides valuable information on the efficacy of XIAP as a neuroprotective agent, but would not likely be applicable to acute human disease. Arterial occlusion in the retina occurs suddenly, often without prior warning, and requires rapid treatment. AAV vectors would be ineffective in a therapeutic strategy unless there was a predisposition to retinal ischemia such as occurs in diabetic retinopathy or there were warning signs of an impending retinal ischemic event. For ischemia, a combination of XIAP protein delivery (for immediate neuroprotection) and XIAP gene therapy (for long term neuroprotection) will be most effective. XIAP protein therapy is described herein.

### EXAMPLE 4

### XIAP Protection in Retinal Detachment

Studies in the laboratory of David Zacks (Kellogg Eye Center, University of Michigan) have shown that following retinal detachment, cell death of the photoreceptors proceeds via apoptosis. Apoptosis peaks at 3 days post detachment. Furthermore, Dr. Zacks has characterized the apoptotic mechanism and shown that cell death proceeds through the activation of caspases 3, 7 and 9. Since XIAP has been shown to efficiently bind to and block the action of these caspases, AAV delivery of XIAP to the photoreceptors should significantly block photoreceptor apoptosis.

Methods

Serotype-5 AAV carrying XIAP under the control of the chicken beta-actin promoter (AAV-CBA-XIAP) and control virus encoding GFP (AAV-CBA-GFP) is injected into the subretinal space of the left eye using the same methods as in Example 2. Thirty animals are injected for each experimental group. Two weeks post-subretinal injection, animals are anaesthetized and their pupils dilated. A sclerotomy is created approximately 2 mm posterior to the limbus with a 20-gauge microvitreoretinal blade. A Glaser subretinal injector (20-gauge shaft with a 32-gauge tip) connected to a syringe filled with 10 mg/mL sodium hyaluronate (Healon; Pharmacia and Upjohn Co.) is introduced into the vitreous cavity. A retinotomy is created in the peripheral retina with the tip of the subretinal injector, and the sodium hyaluronate is injected into the subretinal space, detaching the retina from the underlying RPE. The retinal detachment is induced in the left eye in the same region as the AAV subretinal injection. Right eyes serve as controls.

Ten animals from each of the GFP and XIAP groups are enucleated at each time point (3, 7 and 28 days post detachment). For western blot analysis and caspase assays, five experimentally detached retinas are dissected from the attached portion of the retina and processed. Retinal extracts are tested for caspase 9 activity, Fas/Fas ligand involvement and cytochrome c release. For histological analysis, the five animals undergo cardiac perfusion with 4% formaldehyde in PBS buffer and the eyes removed, processed, embedded in paraffin and sectioned at 6 μm.

Immunohistochemistry with HA antibodies is conducted to confirm XIAP over-expression and TUNEL analysis is performed to examine apoptotic profiles. Hematoxylin and Eosin staining is conducted to examine cell counts in XIAP treated versus control retinas.

### EXAMPLE 5

### AAV with Inducible Promoters in Genetic Models of Retinitis Pigmentosa

Even though AAV vectors are non-pathogenic, problems encountered with other viral gene therapy systems have meant that all viral vectors have to be rigidly tested to ensure safety prior to clinical trials. Inducible vectors have fewer safety concerns because they allow control of gene expression and provide the ability to turn gene expression on or off. Therefore, tight regulation of XIAP gene expression may be desirable.

Methods

The tetracycline-responsive system, originally described by Gossen and Bujard, permits tight regulation of target gene expression under the control of oral delivery of the drug doxycycline, an analog of tetracycline. McGee Sanftner et al. have made modifications to this system and created recombinant AAV vectors which they have used to effectively target photoreceptors. The tet-inducible system involves two AAV vectors. The first vector contains the inducible promoter which consists of seven tandem copies of the tet operator sequence upstream of a minimal CMV promoter driving target expression. The second vector contains a transcriptional silencer and a transcriptional activator which have opposite responses to doxycycline. In the presence of doxycycline, the activator binds the inducible promoter and induces expression of the target gene. In the absence of doxycycline, the silencer binds the inducible promoter and blocks expression. McGee Sanftner et al. have shown that the ratios of the two vectors and the dosage of doxycycline can be varied to optimise gene expression. Once these vectors are optimised, they are tested in wild-type Sprague Dawley rats. We subretinally inject the two AAV vectors, as previously described, testing different combinations of the two vectors and different doses of doxycycline in the drinking water. The level of XIAP expression are assessed using western blot analysis with anti-HA antibodies. Once the ideal parameters are determined for optimal XIAP expression, the effects of inducible XIAP on disease progression in the P23H rats are analyzed. Our results to date suggest that XIAP should be effective in halting or delaying disease progression. With the inducible system, we are able to examine the protective effects of different levels of XIAP over-expression by varying the dose of the doxycycline in the drinking water. We are also able to test the effectiveness of delayed intervention by delaying doxycycline administration.

### EXAMPLE 6

### Treatment of Motor Neuron Disease with XIAP Gene Therapy

In spinal muscular atrophy (SMA) the primary cause of motor neuron death is the depletion of SMN protein. To date, SMN has been shown to be involved in several essential cellular processes, none of which have been directly linked to cellular attrition. However, motor neuron death in SMN clearly proceeds through apoptosis; indeed SMA was one of the first examples of the involvement of apoptosis in a pathological process. Preliminary work done by our group has shown that inhibition of apoptosis, using the neuronal IAP (NAIP), can rescue motor neuron degeneration in mild SMA models. XIAP is amenable for future gene therapy applications using recombinant viral vectors and should provide a rescue phenotype given its potent activity.

We are also interested in the treatment of a second motor neuron disease, amyotrophic lateral sclerosis (ALS). 5-10% of ALS cases are hereditary; amongst them, we have shown that ALS2 (a juvenile, autosomal recessive form of ALS) is caused by mutations in alsin, a putative GTPase regulator protein. Like SMA, the pathophysiology of ALS is poorly understood, but again, dying motor neurons exhibit features reminiscent of apoptosis and molecular elements of the programmed cell death machinery are activated in ALS spinal cords. Critically, inhibition of apoptosis by transgenic XIAP in an ALS1 (SOD1) mouse has been shown to attenuate disease progression.

We have obtained two mouse models for SMA, an SMN heterozygote (Smn^{-/+}) that presents a mild phenotype and a severe disease model that is nullizygous for SMN, with a partial rescue provided by a poorly expressing SMN transgene (Smn^{-/-} tgSMN2). Crossing of these mouse strains with the UbcXIAP transgenic mice is done to confirm the usefulness of XIAP in treating SMA. The mild Smn^{-/+} SMA model presents with motor neuron attrition (about 50%) in the lumbar region. We have also observed reduced locomotion and coordination using behavioral testing established in our laboratory, which reflects motor neuron loss. These analyses include assessment of locomotion and coordination, traits known to be altered in individuals with SMA. These tests are thus used to assess the effect of transgenic XIAP on the Smn^{-/+} mice. In all studies, groups of at least four are tested for each gender, age group and genotype. Motor neurons are counted in the spinal cords of XIAP transgenic Smn^{-/+} mice at 5 weeks, 3 months, 6 months and 1 year of age and compared to controls.

Rescue of motor neuron death by XIAP should be accompanied by a decrease in the cleavage and activation of the caspases. Accordingly, we have assessed the expression and activation of the key executioner caspases-3 and -6 in the spinal cords of the Smn^{-/+} mice. Interestingly, we observed no changes in caspase-3, whereas cleaved caspase-6 was increased at 3 months. Caspase-6 has been widely implicated in neuronal cell death in Alzheimer's disease.

The Smn^{-/-} tgSMN2 mice show limited survival (maximum of 5 days). To determine the impact of XIAP over-expression on longevity in this strain, mice are checked twice daily from the time of birth in order to precisely determine any alterations in survival that the addition of a XIAP transgene may impart.

The alsin gene is associated with the ALS phenotype. To date, the murine gene for alsin has been deleted by removing the majority of the coding sequence in two different ES cell lines. No homozygously deleted animals could be generated from one of these lines while hemizygous animals were phenotypically normal. This suggests an essential function for alsin, although it should be noted that this observation was not reproduced with the second ES line, suggesting variable penetrance of the knockout phenotype or of modifying genes. The first ES cell line is used to “knock-in” alsin with the appropriate point mutations, in order to recreate the ALS2 genotype and phenotype for use in this study.

The utility of XIAP gene therapy is assessed in various mouse models. Over the past five years a nonprimate, lentiviral vector system based on minimal EIAV (equine infectious anemia virus) vectors, in which all accessory genes have been removed, has been developed. EIAV vectors pseudotyped with rabies virus glycoprotein (rabies-G) expressing the reporter gene lacZ demonstrate high and sustained (up to 8 months) transduction of neuronal cells, including spinal cord motor neurons in the rat. Moreover, rabies-G pseudotyped vectors are transported in a retrograde fashion from the site of muscle injection to anatomically connected neuronal cell bodies, including motor neurons. To date, a lentiviral vector expressing human XIAP (EIAV-XIAP) has been generated at BioMedica and has been shown to express in culture. To test the efficacy of this approach, we inject EIAV-XIAP (or EIAV-LacZ as control) into the gastrocnemius muscle of wt and Smn^{-/+} mice at 1 month of age (prior to the loss of motor neurons). The mice will be assessed monthly for locomotion and coordination and at one year for motor neuron count. Similar assessments are undertaken in the severe Smn^{-/-} tgSMN2 model.

### EXAMPLE 7

### Treatment of Muscular Dystrophies with XIAP Gene Therapy

Apoptosis plays an important role in the differentiation, development and homeostatic maintenance of skeletal muscle, but the prevailing wisdom has been that fiber death in muscular dystrophy is typically necrotic. However, there is a growing awareness that apoptosis and necrosis are not separable phenomena and that the latter can be one outcome of an initially apoptotic process. For example, in the focal ischemia model of stroke using thread occlusion of the middle cerebral artery, established dogma states that the center of the infarct area dies by necrosis. Surprisingly, however, XIAP is dramatically protective in this model. Recent studies suggest the same situation is true in several muscular dystrophies. Markers of apoptosis and necrosis co-exist in the muscle of both Duchenne muscular dystrophy (DMD) patients and in the mdx mouse model for DMD. In the mdx mouse, an acute, necrotic degenerative phase begins at approximately 3 weeks postnatal and eventually resolves in adult mice. However, apoptotic myonuclei can be detected prior to this phase in otherwise normal fibers, peak at approximately 4 weeks when necrotic fiber death is obvious, and decline thereafter. Apoptosis is energy-dependent and studies have shown that a switch to the passive process of necrosis occurs when ATP is limited, a situation present in damaged DMD muscle fibers due to functional ischemia. Taken together, and by analogy with the focal ischemia model, this suggests that fiber death is initiated through apoptotic pathways but that necrotic degeneration quickly predominates. We therefore believe that XIAP will preserve fiber integrity and function, by delaying the onset of apoptosis, in dystrophic mouse models. The mdx mouse is an example of a relatively slowly progressing disease (in the mouse). Other models (detailed below) can also be tested as examples of dystrophies with varying degrees of severity.

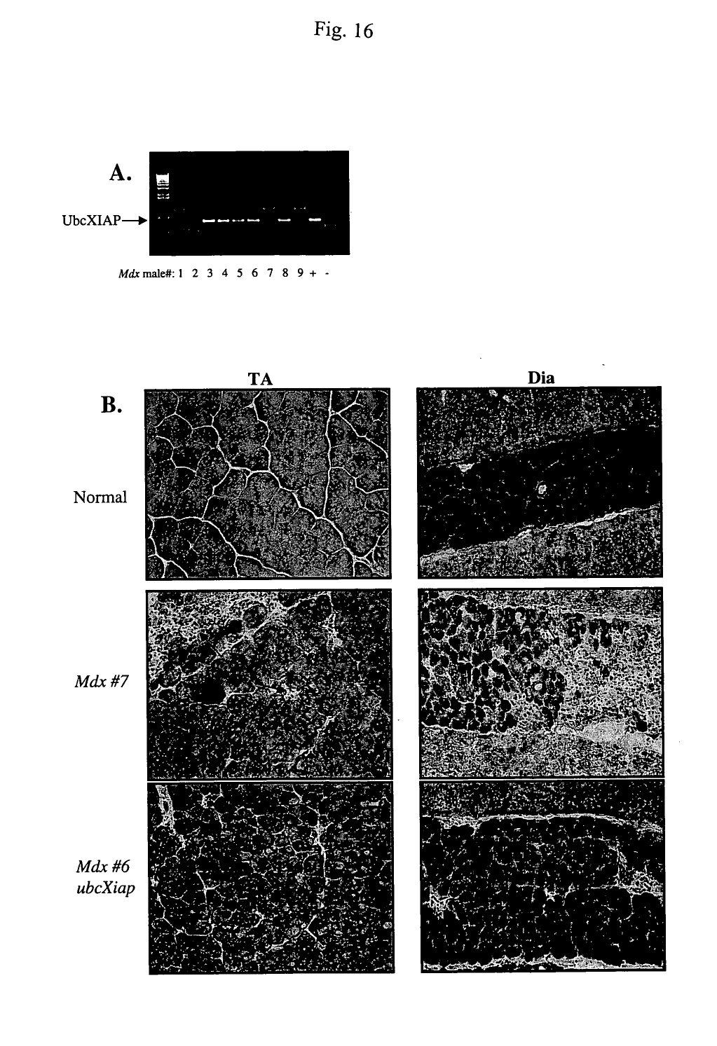

Homozygous mdx females have been crossed to (single copy) UbcXIAP males. To date, 7 F1 males have been scored blind for the degree of fiber degeneration and inflammatory infiltration in three muscle types (Table 1). The preliminary data shows that, overall, the pathology appears ameliorated in those mdx males that inherited the UbcXIAP transgene (FIGS. 16A and 16B; and Table 1). The degree of cellular infiltration is strikingly reduced in these mice. Fiber size and shape also appears more regular. As necrosis is typically focal in dystrophic muscle, cryopreserved sections are stained for the C5b-9 membrane attack complex (MAC), a reliable assay for the detection of dystrophic fibers. The total number of dystrophic fibers per muscle is counted. We have observed strong MAC staining in the TA of non-transgenic mdx mice, but not in their XIAP-transgenic littermates.

TABLE 1
Pathology score of Mdx × UbcXiap mice

| Mouse # | Tg | TA | Diaph. | Gastro. |
|---|---|---|---|---|
| 1 | Y | + | ++ | + |
| 2 | Y | + | ++ | ++ |
| 3 | Y | ++ | ++ | ++ |
| 4 | Y | + | + | + |
| 5 | N | +++ | +++ | +++ |
| 6 | Y | + | ++ | + |
| 7 | N | ++ | +++ | +++ |

+ centronucleation
++ above, plus fiber degeneration, mild inflammation
+++ above plus pronounced cellular infiltration

To further characterize the rescue phenotype, double copy UbcXIAP mice are used to increase XIAP expression to determine if pathology can be further ameliorated. Muscle mass is determined at various times after birth to determine whether the hypertrophy characteristic of mdx muscle has been reversed. Fiber integrity is assessed by serum creatine kinase assays and by the systemic injection of Evans Blue dye (EBD), sensitive measures of muscle membrane integrity. Staining for the developmental MHC isoform is used to score regenerating fibers. The apparent reduction in cellular infiltration is confirmed by staining sections for Mac-1, a marker for several immune cells typically present in mdx muscle.

To further test for functional rescue in older mice, mdx mice exhibit a peak in fiber death between 3 and 10 weeks of age, after which there is functional recovery, but they remain sensitive to exercise-induced damage. Six month-old mice will therefore be injected i.p. with EBD, five hours prior to being placed on an Omnipacer treadmill (Accuscan Instruments) with a downward slope (15 degree downward with increasing speed to 19 m/min) for eccentric exercise. Mice will be sacrificed 24 hours later and GFP expression are quantified as the percentage of the total sectional area that is GFP positive, using Northern Eclipse software (Empix Imaging). To determine whether a reduction in apoptosis indeed underlies the rescue phenotype, mdx/UbcXIAP muscle sections are analyzed by TUNEL staining (ApopTag Plus Peroxidase kit) at one and two weeks post-natal, when apoptosis prior to the onset of necrosis has been documented. Immunohistochemistry for active caspase-3 is also performed.

Two other dystrophic models are crossed to the UbcXIAP mice and analyzed as described above for mdx mice. The δ-dystroglycan knockout mouse is a model for limb-girdle muscular dystrophy (LGMD) type 2F. This mouse presents with a severe, rapidly progressing dystrophy at birth. Apoptosis has not yet been examined in this mouse, but in a related model, γ-sarcoglycan knockout mice (for LGMD2C), abundant apoptotic myonuclei are evident. The calpain-3 knockout mouse is a model for LGMD2A. This mouse presents with a moderate, relatively slowly progressing dystrophy at birth. This mouse is of particular interest as the levels of apoptotic myonuclei in LGMD2A are considerably higher than in any other disease.

We have AAV5 vectors expressing myc-tagged XIAP (AAV-XIAP) or GFP (AAV-GFP), under the control of the strong chicken β-actin promoter (CBA). The more recently characterized AAV5 serotype displays greatly enhanced tropism for skeletal muscle over other commonly used serotypes, such as AAV2. Neonatal mice are injected in the hindlimb according to established protocol; preliminary experiments to determine the baseline inoculum necessary to achieve maximal, sustained XIAP expression are carried out first in wild-type mice. Expression is determined by staining of sectioned tissues and by immunoblot. The basic approach required to establish the rescue of dystrophic muscle does not vary from that described for the transgenic approach.

### EXAMPLE 8

### Treatment of Skeletal Muscle Atrophy with XIAP Gene Therapy

Skeletal muscle atrophy is a debilitating consequence of many diverse pathological conditions, such as aging, cancer, HIV/AIDS, bed-rest, spinal cord injury, neuromuscular disease, and spaceflight. Muscle atrophy occurs when the rate of muscle protein breakdown exceeds that of muscle protein synthesis, and net muscle protein loss ensues. Ultimately, net muscle protein loss leads to smaller muscle fibers that have, among other consequences, a diminished capacity to generate force and power and to clear glucose and fats from circulation. Not surprisingly, such consequences have major health implications. For instance, diminished capacity to generate force and power that occurs with aging increases the incidence of falls and fractures in the elderly. Furthermore, decreased capacity to clear glucose and fats is a major cause of obesity, type II diabetes and cardiovascular disease. These few examples clearly illustrate the individual and societal costs of muscle atrophy. Accordingly, a major directive of muscle physiologists in recent years has been to elucidating the underlying cellular and molecular mechanisms that mediate muscle atrophy with the hope of identifying therapeutically relevant targets.

The pro-apoptotic protein caspase-3 has emerged as one such mediator of skeletal muscle atrophy. In a series of recent papers, several groups have demonstrated that caspase-3 activation is an initiating step in myofibrillar protein degradation during muscle atrophy. In response to various atrophic stimuli, caspase-3 is activated and cleaves various proteins from the highly structured sarcomeres, which allows them to be ubiquitin-tagged and degraded in the proteasome. Importantly, caspase-3 repression using synthetic caspase-3 inhibitors was shown to attenuate muscle atrophy in vitro and in vivo.

Given that (i) caspase-3 activation occurs during and is required for skeletal muscle atrophy, and (ii) XIAP is a potent inhibitor of caspase-3 activation, we performed experiments designed to test the hypothesis that overexpressing XIAP in skeletal muscle could mitigate against the onset and/or progression of muscle atrophy.

Methods

To test our hypothesis, we used an in vitro C2C12 cell culture model of skeletal muscle atrophy. We cultured C2C12 myoblasts (ATCC) until confluent, and induced myoblast differentiation into myotubes by switching media supplement from 10% fetal bovine serum (growth media) to 2% horse serum (differentiation media). After 4 days of differentiation, by which time the majority of myoblasts had differentiated and fused into myotubes, we added adenoviruses to the media carrying plamids encoding for XIAP or a GFP or LacZ control. After 24 h, we washed off the media and switched to serum-free media (starvation media) to model starvation-induced muscle atrophy. We took pictures and collected protein and RNA samples at 24 h after inducing starvation, and assessed: (a) mean myotube diameter using computerized software; (b) total protein per plate using a Lowry assay; (c) total RNA per plate using the UV spectrophotometer; (d) mRNA expression of the muscle-specific E3 ubiquitin ligases muscle ring finger 1 (MURF-1) and atrogin-1, which are both markers of muscle atrophy. Additionally, we performed similar experiments in which we collected data throughout a timecourse within the first 24 h of starvation.

Results

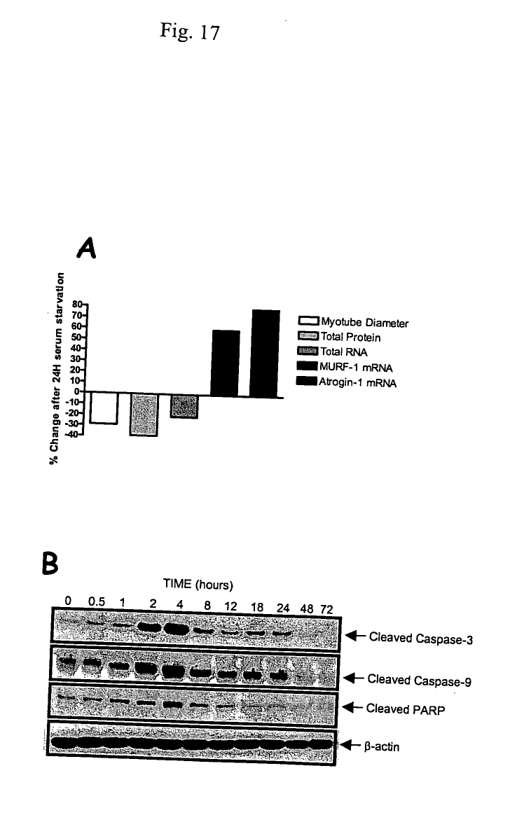

In the in vitro model skeletal muscle cell atrophy, serum starvation for 24 h induced a 29% decrease in mean myotube diameter, which was accompanied by a 39% decrease in total protein per plate, and a 21% decrease in total RNA per plate (FIG. 17A). Additionally, serum-starvation led to a 1.6- and 1.8-fold increase in MURF-1 and Atrogin-1 mRNA expression, respectively (FIG. 17A). Importantly, serum starvation led to rapid caspase-3 activation, as well as the upstream initiator caspase-9 and the apoptotic marker PARP (FIG. 17B), confirming in our hands that these proteins respond to atrophic stimuli in cultured muscle.

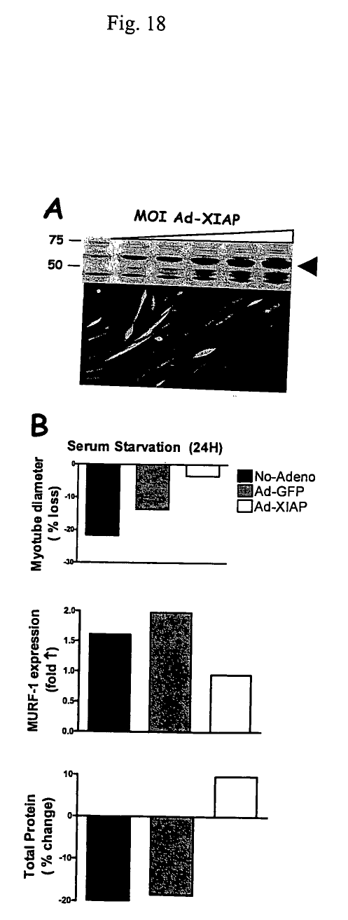

Importantly, our experiments using adenovirus-mediated XIAP overexpression strongly support our hypothesis that XIAP overexpression can protect C2C12 myotubes and, by extension, skeletal muscle, from starvation-induced atrophy (FIGS. 18A and 18B). Ad-XIAP treatment for 24 h led to a dose-dependent increase in XIAP expression in fully differentiated myotubes (FIG. 18A, top panel). Importantly, XIAP overexpression (MOI 50) prior to serum-starvation protected myotubes from various markers of atrophy when compared to no adenovirus or Ad-GFP. Specifically, XIAP overexpression largely attenuated the decrease in myotube diameter following starvation, and completely prevented the increase in MURF-1 mRNA expression and the loss of total protein that accompanies starvation.

### EXAMPLE 9

### XIAP Over-Expression Improves Dopamine Graft Survival and Function in the 6-hydroxydopamine Rat Model of Parkinson's Disease

General use of neurotransplantation as a treatment for Parkinson's disease (PD) is limited by the poor survival of implanted embryonic dopamine neurons that occurs in two phases. In the initial phase following transplantation, a large percentage of neurons (90%) are lost within the first few days. Several lines of evidence suggest that apoptotic mechanisms involving activation caspase-3 and c-Jun-N-terminal kinase (JNK) signaling pathways contribute to the death of grafted fetal dopamine neurons shortly after implantation. Both of these cell death pathways are blocked by the anti-apoptotic protein XIAP. We have previously demonstrated that over-expression of XIAP reduces dopamine neuron cell loss in animal models of Parkinson's disease. Based on the ability of XIAP over-expression to protect dopamine neurons in these animal models of Parkinson's disease, we predict that XIAP over-expression in the transplanted cells will reduce this immediate cell loss.

The second or delayed phase of dopamine neuron loss is mediated by immune-mediated rejection of the graft. Rats implanted in the striatum with ventral mesecenphalic (VM) dopamine cells from embryonic mice (xenogenic grafts) reject the graft unless immunosuppressed. Immunohistochemical analysis of xenogenic grafts following cessation of immunosuppressive therapy show rapid development of a marked microglial response in the graft. This inflammatory response to a dopaminergic graft in the striatum reduces graft survival and function. It has been hypothesized that similar mechanisms may be responsible for the failure of two recent NIH-sponsored double blind clinical trials to demonstrate the efficacy of this approach. XIAP prevents the death of cells exposed to high levels of inflammatory mediators derived from microglia such as TNFα, interleukin-1β (IL-1β) and free radicals (NO* or nitric oxide free radical), suggesting that over-expression of this anti-apoptotic protein may improve the survival of dopaminergic grafts by resisting inflammatory attack. Furthermore, by preventing apoptotic death, XIAP may inhibit activation of a feed forward loop that would otherwise result in a full scale mobilization of the immune system. We show here that fetal dopamine neuron grafts derived from the VM of mice that over-express XIAP (UBC-XIAP) appear to survive in greater number compared to grafts derived from WT animals in the striatum of rats that have sustained a 6-hydroxydopamine (6-OHDA) lesion of the mesostriatal pathway. Second, we show here that surviving XIAP over-expressing dopamine neurons operate properly as assessed by a reduction in the rotational response to amphetamine of 6-OHDA lesioned rats transplanted with embryonic (E18) ventral mesencephalic (VM) dopamine neurons from UBC-XIAP mice relative to wild-type (WT) littermates.

Methods

Experimental Protocol

Female Wistar rats (200-225 g) received two stereotaxic injections of 6-OHDA into the right ascending mesostriatal dopaminergic pathway under anaesthesia. After a two week recovery period, all rats were given an amphetamine injection (5 mg/kg i.p.) and their rotational scores recorded over a 60 min period. Only animals exhibiting a mean ipsilateral rotation score of 8 or more complete body turns/min that did not demonstrate gross motor or dietary impairments will be used for transplantation. Transplantation occurred the next day. Two groups of 6-OHDA lesioned rats received intra-striatal injections of embryonic VM dopamine neurons derived from either UBC-XIAP mice or WT littermates. Three weeks later, the rotational response to amphetamine (5 mg/kg, i.p.) was assessed in these two groups. Following rotation tests, animals were sacrificed and brains processed to visualize surviving tyrosine hydroxylase (TH) immunoreactive dopamine neurons grafted into the 6-OHDA-denervated striatum.

Microtransplantation: Microtransplantation minimizes injury to the host tissue results in a precise and reproducible implantation of neuronal cells into small brain structures. Briefly, the microtransplantation technique involves stereotactic injection of nanoliter volumes of single cell suspensions prepared from VM tissue of 13-day old mouse fetuses into the host striatum of 6-OHDA lesioned animals. To obtain sufficient working volumes of cell suspensions, 25-30 mouse VMs are dissected in DMEM and dissected tissue is incubated in 0.1% trypsin/0.05% DNAse/DMEM at 37° C. for 20 minutes, rinsed 4 times in 0.05% DNAse/DMEM. Incubated tissue is then mechanically dissociated until a milky, homogenous single cell suspension is achieved. Final cell concentration of approximately 200,000 cell/μl is used with viability >98% as determined by the trypan blue dye exclusion method. All animals receive approximately 100,000 cells during grafting.

UBC-XIAP mice: Despite expressing high levels of XIAP, UBC-XIAP mice are healthy, fertile and live a normal life span. In only one of these mice was a single sporadic tumor observed, suggesting that prolonged XIAP over-expression is a safe means by which to inhibit apoptosis.

Results

XIAP Improves Dopamine Graft Survival

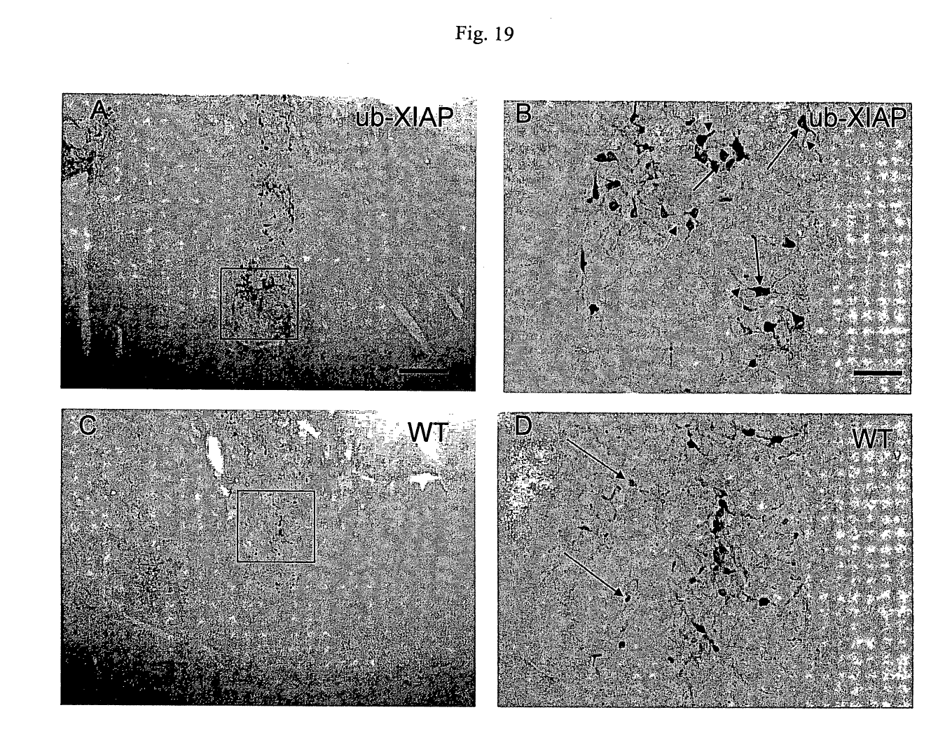

We compared graft survival of embryonic VM cell suspensions from WT and UBC-XIAP mice in the 6-OHDA denervated striatum. One group of rats (n=3) received grafts from WT mice while the other were transplanted with fetal dopamine neurons from UBC-XIAP mice. By comparison to grafts from WT animals, grafts derived from UBC-XIAP mice displayed more surviving dopamine neurons that appeared healthy (large cell bodies, extensive dendritic arbors) 3 weeks after implantation (FIGS. 19A-19D).

Over-Expression of UBC-XIAP Promotes Functional Recovery

Both groups of animals displayed similar rotation rats following 6-OHDA-mediated destruction of the mesostriatal pathway (Table 2). Three weeks following neurotransplantation, animals grafted in the 6-OHDA-denervated striatum with VM neurons from WT animals continued to display high rates of ipsilateral rotation following administration of amphetamine. By contrast, 6-OHDA lesioned rats that received intra-striatal grafts of VM neurons from UBC-XIAP animals displayed considerable reduced rotation rates after amphetamine administration indicating that the grafts had survived and restored dopamine neurotransmission to near normal levels in the striatum ipsilateral to the 6-OHDA lesion.

TABLE 2

| | | | 3 wk post- |
|---|---|---|---|
| Group | Subject # | Pre-graft | graft |
| UBC-XIAP | 9 | 11.90 | 0.90 |
| | 21 | 9.30 | 1.60 |
| | 27 | 9.20 | 2.90 |
| Avg (rotations/min) | | 10.13 | 1.80 |
| SD | | 1.53 | 1.01 |
| WT | 15 | 12.80 | 13.00 |
| | 22 | 8.60 | 10.40 |
| | 12 | 14.10 | 11.30 |
| Avg (rotations/min) | | 11.83 | 11.57 |
| SD | | 2.87 | 1.32 |

Mesencephalic dopamine neuron grafts from embryonic UBC-XIAP, but not WT, mice reduced the rotational response of rats with a unilateral 6-hydroxydopamine lesion to a systemic injection of amphetamine (5 mg/kg i.p.).

Neural transplantation of fetal dopamine neurons is an experimental treatment that holds tremendous promise for the treatment of Parkinson's disease. General use of this grafting technique is limited by the poor survival of implanted embryonic dopamine neurons that interfere with the integration and function of surviving dopamine neurons. Ethical concerns about the use of human fetal donors has encouraged the development of xenografting (pig to man) techniques for dopamine cell replacement. Xenografts elicit a vigorous host immune response severely limiting graft survival. Since XIAP has evolved as a natural mechanism by which to protect cells from the injurious effects of inflammatory mediators, we predict that grafts derived from mice that over-express this anti-apoptotic protein will be resistant to graft rejection. Histological analysis of UBC-XIAP mice indicate that prolonged XIAP over-expression does not result in cellular transformation (cancer) suggesting that increasing XIAP levels is a safe means by which to increase graft survival. By improving grafts survival, fewer cells need be implanted resulting in less disruption of the surrounding tissue that will in turn facilitate integration of the graft with the neighboring striatal tissue and restoration of dopaminergic neurotransmission.

In order to facilitate cell transplant into humans, the invention features the treatment of donor cells with XIAP. This can be achieved with ex vivo gene therapy, where donor cells are transfected with vectors constructed to express XIAP. Alternatively, these cells can be treated with protein preparations of XIAP to protect them from apoptosis throughout the transplant procedure.

### EXAMPLE 10

### Cellular Transplantation

Cellular transplants are potentially of enormous therapeutic value in a wide variety of disease and injury states, including, but not limited to, neural stem cells (NSC), neuronal precursor cells, and neurons in Parkinson's disease (see Example 9), beta cells in diabetes, myoblasts in muscular dystrophies and muscle atrophy, and hepatocytes in acute and chronic liver disease. In all cases the potential of cellular transplants is greatly limited by two key factors: 1) loss of cell viability during isolation and implantation procedures and 2) rapid loss of transplanted cells through the action of the innate and adaptive immune responses. Both of these limitations can be ameliorated through the over-expression of XIAP, which protects cells from a variety of immune system mediators of apoptosis, such as Fas receptor ligation, cytokine cocktails, and the perforin/granzyme B pathway. In addition, we previously showed that XIAP over-expression protects grafted pancreatic beta cells from the immune response, which appears to result in a peripheral tolerance to the graft. XIAP over-expression can thus be used to suppress donor cell death (NSCs, beta cells, myoblasts, hepatocytes) during and shortly after the engraftment procedure, and to induce a tolerance to the grafted cells such that immunosuppression is not required.

Hepatocytes

To demonstrate that XIAP over-expression renders hepatocytes resistant to cytotoxic T cell killing, cells are co-cultured with BALB/c allogeneic lymphocytes that have been activated in vitro by irradiated C57B1/6 lymphocytes. The release of lactate dehydrogenase (Promega Cytotox 96) is used to determine the degree of CTL-mediated killing. CTL proliferation/survival rates are assessed by WST-1 assay and flow cytometry. We hypothesize that abortive CTL killing will result in the death of the effector cells, constituting an in vitro analogy to tolerance in a whole-animal system.

To further demonstrate induction in vivo, hepatocytes are isolated from transgenic animals (UbcXIAP/GFP or UbcGFP) and injected into the tail vein of recipient mice. This is performed first in C57B1/6 control matched animals to determine the seeding frequency of the liver in the absence of an immune reaction, before testing unmatched BALB/c mice. Livers are harvested and sectioned at day 2, day 10 and day 60 post-transplantation and GFP positive cells counted. TUNEL staining is used to detect apoptotic cells and caspase-3 activity levels determined for the quantitative assessment of cell death. The degree of tolerance will be assessed using MLR assays in which lymphocytes (splenocytes) from recipient and control (naïve) animals is tested against irradiated donor C57B1/6 lymphocytes. Proliferation rates will be determined by ³H-thymidine incorporation. We predict a decrease in proliferative response as the recipient mice become tolerant to the donor antigens. Tolerance is confirmed by a second round of engrafting. Sixty days after the initial experiments, engrafted mice receive a second injection of hepatocytes (transduced with RFP, as a marker differentiable from GFP). Genuine tolerance is indicated by acceptance of these cells without requirement for exogenous XIAP.

Myoblasts

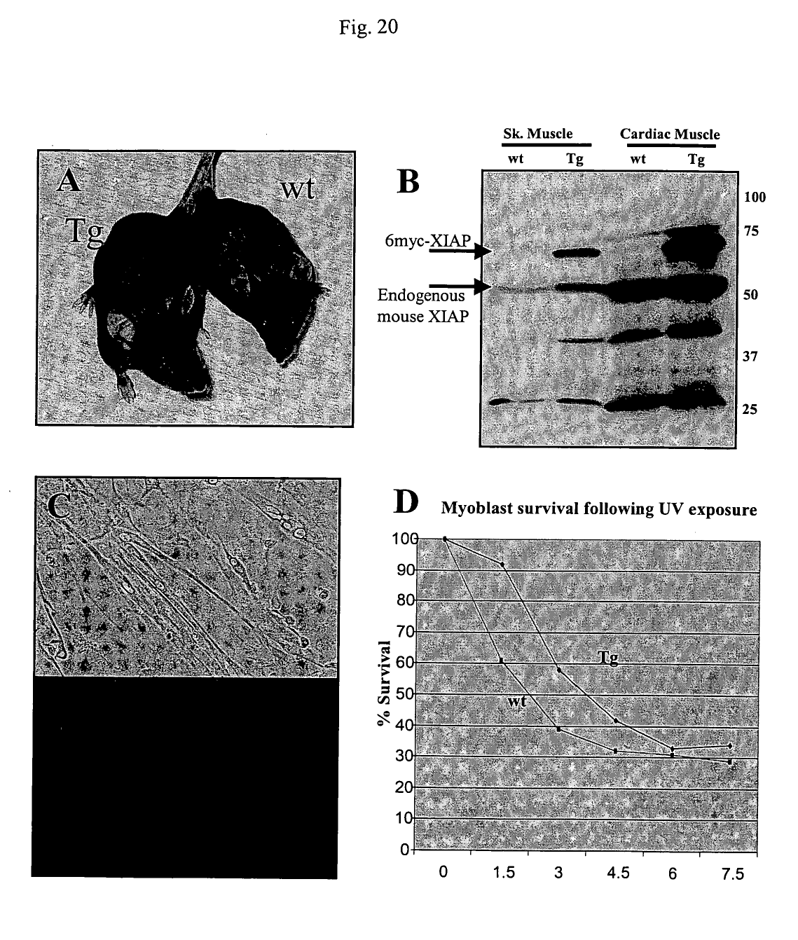

Myoblast transplantation therapy (MTT) may be used for the treatment of DMD and other muscular dystrophies, as well as skeletal muscle atrophy. To demonstrate the feasibility of MTT in the cardiotoxin (CTX) muscle injury model, myoblasts isolated from male donor animals (UbcXIAP/GFP or GFP) are utilized. Skeletal muscle from UbcXIAP mice develops normally (FIG. 20A), indicating that XIAP overexpression (FIG. 20B) will not impair myoblast function or development. Primary myoblast cultures have successfully been established from UbcXIAP mice, differentiate normally in vitro (FIG. 20C) and are more resistant to ultraviolet light induced apoptosis (FIG. 20D), indicating the potential for enhanced survival upon transplantation. These myoblast cultures are co-injected with cardiotoxin directly into the tibialis anterior (TA) muscle of female recipient mice. The concomitant injury to mature muscle fiber allows transplanted myoblasts to contribute to the regenerating muscle mass. This is performed first in C57B1/6 histocompatability (MHC) matched animals to determine whether XIAP over-expression results in a greater graft success rate in the absence of immune system recognition. Animals are euthanized at appropriate intervals for cellular, biochemical, and histological analysis (H&E, TUNEL staining, active caspase-3 immunohistochemistry, GFP expression levels and distribution). In addition, quantification of myonuclei derived from the grafted cells is made by quantitative real-time PCR (Taqman) using male-specific probes and primers.

Myoblast injection into BALB/c animals (histocompatibility unmatched) is performed as described above. NK and CTL infiltration are assessed by additional immunohistochemical staining with surface antigen (CD) specific antibodies. The degree of tolerance is again assessed using MLR assays. We predict that muscle sections from mice that received the UbcXIAP/GFP MTT retain GFP positive fibers, display lower levels of lymphocyte infiltration into the grafted muscle mass, and exhibit reduced or absent markers of apoptosis. Furthermore, we expect a decrease in lymphocyte proliferative responses as the recipient mice become tolerant to the donor antigens.

In addition to myoblasts, bone marrow stem cells (BMSCs) or muscle side population cells can treated with XIAP to prevent apoptosis during cell transplantation.

Other Cell Types

The invention encompasses the XIAP mediated protection from apoptosis in numerous other cell types. These cells include stellate cells, bronchial epithelial cells, bonchial smooth muscle cells, alveolar type II cells, alveolar macrophages, fibroblasts, pulmonary artery smooth muscle cells, pulmonary artery endothelial cells, nasal epithelial cells, keratinocytes, microvascular endothelial cells, Islets of Langerhans, beta cells, kidney proximal tubule cells, kidney cortical epithelial cells, bladder detrusor smooth muscle cells, foreskin fibroblasts, prostate fibroblasts, colon smooth muscle cells, colon epithelial cells, monocytes, polymorphonuclear cells, neutrophils, lymphocytes, eosinophils, splenocytes, cerebral artery endothelial cells, and pituitary cells.

### OTHER EMBODIMENTS

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed is:

1. A method of treating retinal degeneration in a patient in need thereof, said method comprising administering to said patient an expression vector encoding XIAP, wherein said XIAP is positioned in said vector for expression in the retina of said patient.

2. The method of claim 1, wherein said vector is an adeno-associated virus (AAV).

3. The method of claim 1, wherein said XIAP is human XIAP.

4. The method of claim 1, wherein said retinal degeneration is a degeneration of photoreceptors.

5. The method of claim 1, wherein said retinal degeneration is a degeneration of retinal ganglion cells, amacrine cells, bipolar cells, or horizontal cells.

6. The method of claim 1, wherein said patient has been diagnosed with retinitis pigmentosa, glaucoma, age-related macular degeneration, retinal detachment, or retinal ischemia.

7. The method of claim 1 wherein said expression vector is delivered through intravenous, intraarterial, intraocular, intravitreal, subretinal, or transsceleral modes of injection.

8. A method of treating spinal muscular atrophy (SMA), spinobulbar muscular atrophy (SBMA), or amyolateral sclerosis (ALS) in a patient in need thereof, said method comprising administering to said patient an expression vector encoding XIAP, wherein said XIAP is positioned in said vector for expression in the motor neurons of said patient.

9. The method of claim 8, wherein said vector is an AAV or a lentiviral vector.

10. The method of claim 8, wherein said XIAP is human XIAP.

11. A method of treating muscular dystrophy or skeletal muscle atrophy in a patient in need thereof, said method comprising administering to said patient an expression vector encoding XIAP, wherein said XIAP is positioned in said vector for expression in the skeletal muscle cells of said patient.

12. The method of claim 11, wherein said muscular dystrophy is Duchenne muscular dystrophy, Becker muscular dystrophy, Emery-Dreifuss muscular dystrophy, limb girdle muscular dystrophy, myotonic dystrophy, oculopharyngeal muscular dystrophy, or distal muscular dystrophy.

13. The method of claim 11, wherein said vector is an AAV.

14. The method of claim 11, wherein said XIAP is human XIAP.

15. A method of reducing cell death during transplantation of cells, said method comprising expressing XIAP in said cells at a level and for a duration sufficient to reduce cell death during transplantation.

16. The method of claim 15, wherein said cells are selected from neural stem cells, muscle stem cells, satellite cells, liver stem cells, hematopoietic stem cells, bone marrow stromal cells, epidermal stem cells, embryonic stem cells, mesenchymal stem cells, umbilical cord stem cells, precursor cells, muscle precursor cells, myoblast, cardiomyoblast, neural precursor cells, glial precursor cells, neuronal precusor cells, hepatoblasts, neurons, oligodendrocytes, astrocytes, Schwann cells, skeletal muscle cells, cardiomyocytes, and hepatocytes.

17. A method of treating Parkinson's disease in a patient in need thereof, said method comprising administering cells capable of differentiating as dopaminergic neurons, said cells expressing recombinant XIAP, into the patient under conditions that treat said Parkinson's disease.

18. A method of treating muscular dystrophy or skeletal muscle atrophy in a patient in need thereof, said method comprising transplanting cells capable of differentiating as skeletal muscle cells, said cells expressing recombinant XIAP, into the patient under conditions that treat said muscular dystrophy or skeletal muscle atrophy.

19. The method of claim 18, wherein said muscular dystrophy is Duchenne muscular dystrophy, Becker muscular dystrophy, Emery-Dreifuss muscular dystrophy, limb girdle muscular dystrophy, myotonic dystrophy, oculopharyngeal muscular dystrophy, or distal muscular dystrophy.

20. A method of treating cardiac injury in a patient in need thereof, said method comprising transplanting cardiomyocytes or cells capable of differentiating as cardiomyocytes, said cells expressing recombinant XIAP, into the patient under conditions that treat said cardiac injury.

21. A method of treating liver disease in a patient in need thereof, said method comprising transplanting hepatocytes or cells capable of differentiation into hepatocytes, said transplanted cells capable of expressing recombinant XIAP, into the patient under conditions that treat said liver disease.

22. The method of claim 21, wherein said liver disease is acute liver disease or chronic liver disease.

23. A method of treating retinal degeneration in a patient in need thereof, said method comprising administering to said patient a protein preparation of XIAP, wherein said preparation can enter retinal cells.

24. The method of claim 23, wherein said preparation is a protein transduction domain-XIAP fusion.

25. The method of claim 23, wherein said protein transduction domain comprises the Tat, Antp, or VP22.

26. The method of claim 23, wherein said XIAP is human XIAP.

27. The method of claim 23, wherein said retinal degeneration is a degeneration of photoreceptors.

28. The method of claim 23, wherein said retinal degeneration is a degeneration of retinal ganglion cells, amacrine cells, bipolar cells, or horizontal cells.

29. The method of claim 23, wherein said patient has been diagnosed with retinitis pigmentosa, glaucoma, age-related macular degeneration, retinal detachment, or retinal ischemia.

30. The method of claim 23 wherein said protein preparation is delivered through intravenous, intraarterial, intraocular, intravitreal, subretinal, or transsceleral modes of injection.

31. A method of treating SMA1, SMA2, SBMA, or ALS in a patient in need thereof, said method comprising administering to said patient a protein preparation of XIAP.

32. The method of claim 31, wherein said protein preparation is a protein transduction domain-XIAP fusion.

33. The method of claim 31, wherein said protein transduction domain comprises the Tat, Antp, or VP22.

34. The method of claim 31, wherein said XIAP is human XIAP.

35. A method of treating muscular dystrophy or skeletal muscle atrophy in a patient in need thereof, said method comprising administering to said patient a protein preparation of XIAP.

36. The method of claim 35, wherein said muscular dystrophy is Duchenne muscular dystrophy, Becker muscular dystrophy, Emery-Dreifuss muscular dystrophy, limb girdle muscular dystrophy, myotonic dystrophy, oculopharyngeal muscular dystrophy, or distal muscular dystrophy.

37. The method of claim 35, wherein said protein preparation is a protein transduction domain-XIAP fusion.

38. The method of claim 35, wherein said protein transduction domain comprises the Tat, Antp, or VP22.

39. The method of claim 35, wherein said XIAP is human XIAP.

40. The method of claim 35, wherein said protein preparation is delivered through intravenous, intraarterial, or intramuscular modes of injection.

41. A method of reducing cell death during transplantation of cells, said method comprising treating said cells with a protein preparation of XIAP at a level and for a duration sufficient to reduce cell death during transplantation.

42. The method of claim 41, wherein said cells are selected from neural stem cells, muscle stem cells, satellite cells, liver stem cells, hematopoietic stem cells, bone marrow stromal cells, epidermal stem cells, embryonic stem cells, mesenchymal stem cells, umbilical cord stem cells, precursor cells, muscle precursor cells, myoblast, cardiomyoblast, neural precursor cells, glial precursor cells, neuronal precusor cells, hepatoblasts, neurons, oligodendrocytes, astrocytes, Schwann cells, skeletal muscle cells, cardiomyocytes, and hepatocytes.

43. A method of treating Parkinson's disease in a patient in need thereof, said method comprising administering cells capable of differentiating as dopaminergic neurons, said cells being treated with a protein preparation of XIAP, into the patient under conditions that treat said Parkinson's disease.

44. A method of treating muscular dystrophy or skeletal muscle atrophy in a patient in need thereof, said method comprising transplanting cells capable of differentiating as skeletal muscle cells, said cells being treated with a protein preparation of XIAP, into the patient under conditions that treat said muscular dystrophy or skeletal muscle atrophy.

45. The method of claim 44, wherein said muscular dystrophy is Duchenne muscular dystrophy, Becker muscular dystrophy, Emery-Dreifuss muscular dystrophy, limb girdle muscular dystrophy, myotonic dystrophy, oculopharyngeal muscular dystrophy, or distal muscular dystrophy.

46. A method of treating cardiac injury in a patient in need thereof, said method comprising transplanting cardiomyocytes or cells capable of differentiating as cardiomyocytes, said cells being treated with a protein preparation of XIAP, into the patient under conditions that treat said cardiac injury.

47. A method of treating liver disease in a patient in need thereof, said method comprising transplanting hepatocytes or cells capable of differentiation into hepatocytes, said transplanted cells being treated with a protein preparation of XIAP, into the patient under conditions that treat said liver disease.

48. The method of claim 47, wherein said liver disease is acute liver disease or chronic liver disease.
